(19) United States
(12) Patent Application Publication
(10) Pub. No.: US 2010/0166709 A1
(43) Pub. Date: Jul. 1, 2010

(54) Novel Bacteriophage and Antibacterial Composition Comprising the Same

(75) Inventors: In Hye Kang, Gyeonggi-do (KR); Min Tae Park, Seoul (KR); Young Wook Cho, Seoul (KR); Soo An Shin, Seoul (KR); Hyang Choi, Gyeonggi-do (KR)

Correspondence Address: STERNE, KESSLER, GOLDSTEIN & FOX P.L.L.C., 1100 NEW YORK AVENUE, N.W., WASHINGTON, DC, 20005, US

(73) Assignee: CJ Cheiljedang Corporation, Seoul (KR)

(21) Appl. No.: 12/645,414

(22) Filed: Dec. 22, 2009

(30) Foreign Application Priority Data
Dec. 24, 2008 (KR) 10-2008-0133908

(51) Int. Cl. A61K 35/76; A61P 31/04; C12N 7/00

(52) U.S. Cl. 424/93.6; 435/235.1

(57) ABSTRACT

The present invention relates to a novel bacteriophage, more particularly, a bacteriophage that has a specific bactericidal activity against one or more Salmonella bacteria selected from the group consisting of Salmonella Enteritidis, Salmonella Typhimurium, Salmonella Gallinarum, and Salmonella Pullorum. Further, the present invention relates to a composition for the prevention or treatment of infectious diseases including salmonellosis and Salmonella food poisoning caused by Salmonella Enteritidis or Salmonella Typhimurium, Fowl Typhoid caused by Salmonella Gallinarum, and Pullorum disease caused by Salmonella Pullorum, comprising the bacteriophage as an active ingredient. Furthermore, the present invention relates to a feed additive, drinking water, a cleaner and a sanitizer, comprising the bacteriophage as an active ingredient.

### CROSS REFERENCE TO RELATED APPLICATIONS

This application claims the benefit of Korean Patent Application No. 10-2008-0133908, filed Dec. 24, 2009, which is herein incorporated by reference in its entirety.

### REFERENCE TO SEQUENCE LISTING SUBMITTED ELECTRONICALLY VIA EFS-WEB

The content of the electronically submitted sequence listing (Name: SEQLIST; Size: 7,784 bytes; and Date of Creation: Dec. 21, 2009) filed herewith the application is incorporated herein by reference in its entirety.

### BACKGROUND OF THE INVENTION

1. Field of the Invention

The present invention relates to a novel bacteriophage, more particularly, a bacteriophage that has a specific bactericidal activity against one or more Salmonella bacteria selected from the group consisting of Salmonella Enteritidis, Salmonella Typhimurium, Salmonella Gallinarum, and Salmonella Pullorum. Further, the present invention relates to a composition for the prevention or treatment of infectious diseases including salmonellosis and Salmonella food poisoning caused by Salmonella Enteritidis or Salmonella Typhimurium, Fowl Typhoid caused by Salmonella Gallinarum, and Pullorum disease caused by Salmonella Pullorum, comprising the bacteriophage as an active ingredient. Furthermore, the present invention relates to a feed additive, drinking water, a cleaner and a sanitizer, comprising the bacteriophage as an active ingredient.

2. Background Art

Salmonella is a genus of the family Enterobacteriaceae, characterized as Gram-negative, facultatively anaerobic, non spore-forming, rod-shaped bacteria, and most strains are motile by flagella. Salmonella has an average genome GC content of 50-52%, which is similar to those of Escherichia coli and Shigella. The genus Salmonella is a pathogenic microorganism that causes infections in livestock as well as in human. Salmonella enterica, a species of Salmonella bacterium, has a variety of serovars including Gallinarum, Pullorum, Typhimurium, Enteritidis, Typhi, Choleraesuis, and derby (Bopp C A, Brenner F W, Wells J G, Strokebine N A. Escherichia, Shigella, Salmonella. In Murry P R, Baron E J, et al eds Manual of Clinical Microbiology. 7th ed. Washington D.C. American Society for Microbiology 1999; 467-74; Ryan K J. Ray C G (editors) (2004). Sherris Medical Microbiology (4th ed). McGraw Hill. ISBN 0-8385-8529-9.). Of them, Salmonella Gallinarum and Pullorum are fowl-adapted pathogens, Salmonella Typhi is a human-adapted pathogen, Salmonella Choleraesuis and Salmonella derby are swine-adapted pathogens, and Salmonella Enteritis and Salmonella Typhimurium are pathogenic for human and animals. Each serovar causes illness in the respective species, resulting in tremendous damage to farmers or consumers.

A disease of domestic birds caused by Salmonella bacterium is Fowl Typhoid (FT), which is caused by a pathogen, Salmonella Gallinarum (hereinbelow, designated as SG). Fowl Typhoid (FT) is a septicemic disease of domestic birds such as chicken and turkey, and the course may be acute or chronic with high mortality. Recently, it has been reported that Fowl Typhoid frequently occurs in Europe, South America, Africa, and South-East Asia, and damages are increasing. Outbreaks of FT in Korea have been reported since 1992 and economic losses caused by FT in brown, egg-laying chickens are very serious (Kwon Yong-Kook. 2000 annual report on avian diseases. Information publication by National Veterinary Research & Quarantine Service. March, 2001; Kim Ae-Ran et al., The prevalence of Pullorum disease-fowl typhoid in grandparent stock and parent stock in Korea, 2003, Korean J Vet Res(2006) 46(4): 347˜353).

Pullorum Disease is also caused by one of Salmonella bacteria, Salmonella Pullorum (hereinbelow, designated as SP). Pullorum disease occurs in any age or season, but young chickens are particularly susceptible to the disease. During the past century, it has been a serious disease among young chickens at 1-2 weeks of age or younger. Since the 1980s, the occurrence has greatly decreased. However, it has been growing since the middle of the 1990s (Kwon Yong-Kook. 2000 annual report on avian diseases. Information publication by National Veterinary Research & Quarantine Service. March, 2001; Kim Ae-Ran et al., The prevalence of Pullorum disease-fowl typhoid in grandparent stock and parent stock in Korea, 2003, Korean J Vet Res(2006) 46(4): 347˜353).

In Korea, outbreaks of Fowl Typhoid and Pullorum disease have been increasing since the 1990s, inflicting economic damages upon farmers. For this reason, a live attenuated SG vaccine has been used in broilers for the prevention of Fowl Typhoid from 2004 (Kim Ae-Ran et al., The prevalence of Pullorum disease-fowl typhoid in grandparent stock and parent stock in Korea, 2003, Korean J Vet Res(2006) 46(4): 347˜353). Its efficacy is doubtful, and the live vaccine is not allowed to be used for layers because of the risk of egg-transmitted infections. Unfortunately, there are still no commercially available preventive strategies against Pullorum disease, unlike Fowl Typhoid. Thus, there is an urgent need for new ways to prevent Fowl Typhoid and Pullorum disease.

Meanwhile, Salmonella Enteritidis (hereinbelow, designated as SE) and Salmonella Typhimurium (hereinbelow, designated as ST) are zoonotic pathogens, which show no host specificity, unlike SG or SP (Zoobises Report; United Kingdom 2003).

SE and ST are a cause of salmonellosis in poultry, pigs, and cattle. Salmonellosis, caused by Salmonella bacteria, is an acute or chronic infection of the digestive tract in livestock, and shows the major symptoms of fever, enteritis, and septicemia, occasionally pneumonia, arthritis, abortion, and mastitis. Salmonellosis occurs worldwide, and most frequently during the summer months (T. R. Callaway et al. Gastrointestinal microbial ecology and the safety of the food supply as related to Salmonella. J Anim Sci 2008.86:E163-E172). In cattle, typical symptoms include loss of appetite, fever, dark brown diarrhea or bloody mucous stool. The acute infection in calves leads to rapid death, and the infection during pregnancy leads to fetal death due to septicemia, resulting in premature abortion (www.livestock.co.kr). In pigs, salmonellosis is characterized clinically by three major syndromes-acute septicemia, acute enteritis, and chronic enteritis. Acute septicemia occurs in 2˜4 month-old piglets, and death usually occurs within 2˜4 days after onset of symptoms. Acute enteritis occurs during the fattening period, and is accompanied by diarrhea, high fever, pneumonia, and nervous signs. Discoloration of the skin may occur in some severe cases. Chronic enteritis is accompanied by continuing diarrhea (www.livestock.co.kr).

Once an outbreak of salmonellosis by SE and ST occurs in poultry, pigs, and cattle, it is difficult to cure by therapeutic agents. The reasons are that Salmonella bacteria exhibit a strong resistance to various drugs and live in cells being impermeable to antibiotics upon the occurrence of clinical symptoms. Up to now, there have been no methods for effectively treating salmonellosis caused by SE and ST, including antibiotics (www.lhca.or.kr).

As in livestock, SE and ST cause infections in human via livestock products, leading to Salmonella food poisoning. Consumption of infected, improperly cooked livestock products (e.g., meat products, poultry products, eggs and by-products) infects human. Salmonella food poisoning in human usually involves the prompt onset of headache, fever, abdominal pain, diarrhea, nausea, and vomiting. The symptoms commonly appear within 6-72 hours after the ingestion of the organism, and may persist for as long as 4-7 days or even longer (NSW+HEALTH. 2008.01.14.).

According to a report by the CDC (The Centers for Disease Control and Prevention, USA), 16% of human food poisoning outbreaks between 2005 and 2008 were attributed to Salmonella bacteria, and 20% and 18% of that total was caused by SE and ST, respectively. With respect to Salmonella food poisoning in human between 1973 and 1984, the implicated food vehicles of transmission were reportedly chicken (5%), beef (19%), pork (7%), dairy products (6%), and turkey (9%). In 1974-1984, the bacterial contamination test on broilers during the slaughter process showed 35% or more of Salmonella incidence. In 1983, Salmonella was isolated in 50.6% of chicken, 68.8% of turkey, 60% of goose, 11.6% of pork, and 1.5% of beef. Further, a survey carried out in 2007 reported that Salmonella was found in 5.5% of raw poultry meat and 1.1% of raw pork. In particular, it was revealed that SE commonly originated from contaminated egg or poultry meat, and ST from contaminated pork, poultry meat, and beef (www.cdc.gov). For example, food poisoning caused by SE has rapidly increased in the US, Canada, and Europe since 1988, and epidemiological studies demonstrated that it was attributed to eggs or egg-containing foods (Agre-Food Safety Information Service(AGROS). Domestic and foreign food poisoning occurrence and management trend. 2008. 02). A risk assessment conducted by FAO and WHO in 2002 noted that the human incidence of salmonellosis transmitted through eggs and poultry meat appeared to have a linear relationship to the observed Salmonella prevalence in poultry. This means that, when reducing the prevalence of Salmonella in poultry, the incidence of salmonellosis in humans will fall (Salmonella control at the source; World Health Organization. International Food Safety Authorities Network (INFOSAN) Information Note No. 03/2007). Recently, fears about food safety have been spurred by outbreaks of salmonella from products as varied as peanuts, spinach, tomatoes, pistachios, peppers and, most recently, cookie dough (Jane Black and Ed O'Keefe. Overhaul of Food Safety Rules in the Works. Washington Post Staff Writers Wednesday, Jul. 8, 2009).

For these reasons, Salmonella infections must be reported in Germany (§6 and §7 of the German law on infectious disease prevention, Infektionsschutzgesetz). Between 1990 and 2005 the number of officially recorded cases decreased from approximately 200,000 cases to approximately 50,000. It is estimated that every fifth person in Germany is a carrier of Salmonella. In the USA, there are approximately 40,000 cases of Salmonella infection reported each year (en.wikipedia.org/wiki/Salmonella#cite_note-2).

Therefore, there is an urgent need to control SE and ST, which cause salmonellosis in livestock and human. The collaborative efforts of USDA and FDA have developed a number of effective strategies to prevent salmonellosis that causes over 1 million cases of food-borne illness in the United States. Among them is a final rule, issued by the FDA, to reduce the contamination in eggs. The FDA will now require that egg producers test regularly for lethal Salmonella during egg production, storage and shipment. As a result, an estimated 79,000 illnesses and 30 deaths due to contaminated eggs will be avoided each year (Jane Black and Ed O′Keefe. Overhaul of Food Safety Rules in the Works. Washington Post Staff Writers Wednesday, Jul. 8, 2009). In Denmark, conservative estimates from a cost benefit analysis comparing Salmonella control costs in the production sector with the overall public health costs of salmonellosis suggest that Salmonella control measures saved the Danish economy US$ 14.1 million in the year 2001 (Salmonella control at the source. World Health Organization. International Food Safety Authorities Network(INFOSAN) Information Note No. 03/2007).

Meanwhile, bacteriophage is a specialized type of virus that only infects and destroys bacteria, and can self-replicate only inside host bacteria. Bacteriophage consists of genetic material in the form of single or double stranded DNA or RNA surrounded by a protein shell. Bacteriophages are classified based on their morphological structure and genetic material. There are three basic structural forms of bacteriophage according to morphological structure: an icosahedral (twenty-sided) head with a tail, an icosahedral head without a tail, and a filamentous form. Based on their tail structure, bacteriophages having icosahedral head and double-stranded, linear DNA as their genetic material are divided into three families: Myoviridae, Siphoviridae, and Podoviridae, which are characterized by contractile, long noncontractile, and short noncontractile tails, respectively. Bacteriophages having icosahedral head without a tail and RNA or DNA as their genetic material are divided based on their head shape and components, and the presence of shell. Filamentous bacteriophages having DNA as their genetic material are divided based on their size, shape, shell, and filament components (H. W. Ackermann. Frequency of morphological phage descriptions in the year 2000; Arch Virol (2001) 146:843-857; Elizabeth Kutter et al. Bacteriophages biology and application; CRC press).

During infection, a bacteriophage attaches to a bacterium and inserts its genetic material into the cell. After this a bacteriophage follows one of two life cycles, lytic or lysogenic. Lytic bacteriophages take over the machinery of the cell to make phage components. They then destroy or lyse the cell, releasing new phage particles. Lysogenic bacteriophages incorporate their nucleic acid into the chromosome of the host cell and replicate with it as a unit without destroying the cell. Under certain conditions, lysogenic phages can be induced to follow a lytic cycle (Elizabeth Kutter et al. Bacteriophages biology and application. CRC press).

After the discovery of bacteriophages, a great deal of faith was initially placed in their use for infectious-disease therapy. However, when broad spectrum antibiotics came into common use, bacteriophages were seen as unnecessary because of having a specific target spectrum. Nevertheless, the misuse and overuse of antibiotics resulted in rising concerns about antibiotic resistance and harmful effects of residual antibiotics in foods (Cislo, M et al. Bacteriophage treatment of suppurative skin infections. Arch Immunol. Ther. Exp. 1987.2:175-183; Kim sung-hun et al., Bacteriophage; New Alternative Antibiotics. Biological research information center (BRIC)). In particular, antimicrobial growth promoter (AGP), added to animal feed to enhance growth, is known to induce antibiotic resistance, and therefore, the ban of using antimicrobial growth promoter (AGP) has been recently introduced. In the European Union, the use of all antimicrobial growth promoters (AGPs) was banned from 2006. Korea has banned the use of some AGPs from 2009, and is considering restrictions on the use of all AGPs at 2013˜2015.

These growing concerns about the use of antibiotics have led to a resurgence of interest in bacteriophage as an alternative to antibiotics. 7 bacteriophages for control of E. coli 0157:H are disclosed in U.S. Pat. No. 6,485,902 (granted in 2002—Use of bacteriophages for control of Escherichia coli O157). 2 bacteriophages for control of various microorganisms are disclosed in U.S. Pat. No. 6,942,858 (granted by Nymox in 2005). Many companies have been actively trying to develop various products using bacteriophages. EBI food system (Europe) developed a food additive for preventing food poisoning caused by Listeria monocytogenes, named Listex-P100, which is the first bacteriophage product approved by the US FDA. A phage-based product, LMP-102 was also developed as a food additive against Listeria monocytogenes, approved as GRAS (Generally regarded as safe). In 2007, a phage-based wash produced by OmniLytics was developed to prevent E. coli O157 contamination of beef during slaughter, approved by USDA's Food Safety and Inspection Service (FSIS). In Europe, Clostridium sporogenes phage NCIMB 30008 and Clostridium tyrobutiricum phage NCIMB 30008 were registered as a feed preservative against Clostridium contamination of feed in 2003 and 2005, respectively. Such studies show that research into bacteriophages for use as antibiotics against zoonotic pathogens in livestock products is presently ongoing.

However, most of the phage biocontrol studies have focused on the control of E. coli, Listeria, and Clostridium. Salmonella is also a zoonotic pathogen, and damages due to this pathogen are not reduced. As mentioned above, since SE and ST exhibit multiple drug resistance, nationwide antimicrobial resistance surveillance has been conducted in Korea under Enforcement Decree of the Act on the Prevention of Contagious Disease (Executive Order 16961), Enforcement ordinance of the Act on the Prevention of Contagious Disease (Ministry of Health and Welfare's Order 179), and Organization of the National Institute of Health (Executive Order 17164). Accordingly, there is a need for the development of bacteriophages to control Salmonella.

### BRIEF SUMMARY OF THE INVENTION

In order to solve the problems generated by the use of broad spectrum antibiotics, the present inventors isolated a novel Salmonella bacteriophage from natural sources, and identified its morphological, biochemical, and genetic properties. The present inventors found that the bacteriophage has a specific bactericidal activity against Salmonella Enteritidis (SE), Salmonella Typhimurium (ST), Salmonella Gallinarum (SG), and Salmonella Pullorum (SP) without affecting beneficial bacteria, and excellent acid-, heat- and dry-resistance, and thus can be used for the prevention and treatment of livestock salmonellosis and Salmonella food poisoning that are caused by Salmonella Enteritidis or Salmonella Typhimurium, and Fowl Typhoid and Pullorum disease that are caused by Salmonella Gallinarum and Salmonella Pullorum. Also, the bacteriophage according to the present invention can be applied to various products for the control of Salmonella bacteria, including feed additive and drinking water for livestock, barn sanitizers, and cleaners for meat products, thereby completing the present invention.

It is an object of the present invention to provide a novel bacteriophage having a specific bactericidal activity against one or more Salmonella bacteria selected from the group consisting of Salmonella Enteritidis, Salmonella Typhimurium, Salmonella Gallinarum, and Salmonella Pullorum.

It is another object of the present invention to provide a composition for the prevention or treatment of infectious diseases caused by one or more Salmonella bacteria selected from the group consisting of Salmonella Enteritidis, Salmonella Typhimurium, Salmonella Gallinarum, and Salmonella Pullorum, comprising the bacteriophage as an active ingredient.

It is still another object of the present invention to provide a feed additive or drinking water for livestock, comprising the bacteriophage as an active ingredient.

It is still another object of the present invention to provide a sanitizer or cleaner, comprising the bacteriophage as an active ingredient.

It is still another object of the present invention to provide a method for preventing or treating infectious diseases caused by one or more Salmonella bacteria selected from the group consisting of Salmonella Enteritidis, Salmonella Typhimurium, Salmonella Gallinarum, and Salmonella Pullorum using the bacteriophage or the composition comprising the bacteriophage as an active ingredient.

The novel bacteriophage of the present invention has a specific bactericidal activity against one or more Salmonella bacteria selected from the group consisting of Salmonella Enteritidis, Salmonella Typhimurium, Salmonella Gallinarum, and Salmonella Pullorum, and excellent acid-, heat- and dry-resistance. Therefore, the novel bacteriophage can be used for the prevention and treatment of infectious diseases caused by Salmonella Enteritidis, Salmonella Typhimurium, Salmonella Gallinarum, or Salmonella Pullorum, including salmonellosis, Salmonella food poisoning, Fowl Typhoid and Pullorum disease, and also used for the control of Salmonella Enteritidis, Salmonella Typhimurium, Salmonella Gallinarum, and Salmonella Pullorum.

### BRIEF DESCRIPTION OF THE DRAWINGS/FIGURES

FIG. 1 is an electron microscopy photograph of ΦCJ3, in which ΦCJ3 belongs to the morphotype group of the family Myoviridae, characterized by an isometric capsid and a long contractile tail;

FIG. 2 is the result of SDS-PAGE of the isolated bacteriophage ΦCJ3, in which protein patterns of the bacteriophage are shown, major proteins of 45 kDa, 62 kDa and 80 kDa and other proteins of 17 kDa, 28 kDa, 110 kDa, and 170 kDa (See-blue plus 2 prestained-standard (Invitrogen) used as marker);

FIG. 3 is the result of PFGE of the isolated bacteriophage ΦCJ3, showing the total genome size of approximately 158 kbp (5 kbp DNA size standard (Bio-rad) as size marker);

FIG. 4 is the result of PCR, performed by using each primer set of ΦCJ3 genomic DNA, in which (A; PCR amplification using the primer set of SEQ ID NOs. 5 and 6, B; PCR amplification using primer set of SEQ ID NOs. 7 and 8, C; PCR amplification using primer set of SEQ ID NOs. 9 and 10, D; PCR amplification using primer set of SEQ ID NOs. 11 and 12) all of A, B, C and D lanes have a PCR product of approximately 1.0 kbp;

FIGS. 5 to 8 are the result of one-step growth experiment of the bacteriophage ΦCJ3, in which the bacteriophage had the burst size of 2×10² pfu or more in Salmonella Gallinarum, Salmonella Pullorum, Salmonella Typhimurium, and Salmonella Enteritidis;

FIG. 9 is the result of acid-resistance test on the bacteriophage ΦCJ3, showing the number of surviving bacteriophage at pH 2.1, 2.5, 3.0, 3.5, 4.0, 5.5, 6.4, 6.9, 7.4, 8.0, 9.0, in which the bacteriophage ΦCJ3 did not lose its activity until pH 3.5, but completely lost its activity at pH 3.0 or lower, as compared to control;

FIG. 10 is the result of heat-resistance test on the bacteriophage ΦCJ3, showing the number of surviving bacteriophage at 37, 45, 53, 60, 70, 80° C. and a time point of 0, 10, 30, 60, 120 min, in which the bacteriophage ΦCJ3 did not lose its activity even after exposure at 60° C. for 2 hrs, and completely lost its activity after exposure at 70° C. or higher for 10 min; and

FIG. 11 is the result of dry-resistance test on the bacteriophage ΦCJ3, performed by using a spray dryer and adding dextrin and sugar as a stabilizer, in which changes in the titers before and after drying were compared to examine the relative stability, and its activity was decreased to approximately 5×10³.

### DETAILED DESCRIPTION OF THE INVENTION

In accordance with an aspect, the present invention relates to a novel bacteriophage having a specific bactericidal activity against one or more Salmonella bacteria selected from the group consisting of Salmonella Enteritidis, Salmonella Typhimurium, Salmonella Gallinarum, and Salmonella Pullorum, in which the bacteriophage belongs to the morphotype group of the family Myoviridae, and has a total genome size of 157-159 kbp and major structural proteins with the size of 44-46 kDa, 61-63 kDa and 79-81 kDa.

Specifically, the bacteriophage of the present invention has the capability of selectively infecting Salmonella gallinarum, Salmonella pullorum, Salmonella typhimurium, and Salmonella Enteritidis, namely, species specificity.

The bacteriophage of the present invention genetically has a total genome size of 157-159 kbp, preferably about 158 kbp, and may include one or more nucleic acid molecules selected from the group consisting of SEQ ID NOs. 1, 2, 3 and 4 within the entire genome, preferably nucleic acid molecules represented by SEQ ID NOs. 1 to 4 within the entire genome.

When the bacteriophage of the present invention is subjected to PCR using one or more primer sets selected from the group consisting of SEQ ID NOs. 5 and 6, SEQ ID NOs. 7 and 8, SEQ ID NOs. 9 and 10, and SEQ ID NOs. 11 and 12, each PCR product of approximately 1 kbp is given. Preferably, when PCR is performed using all of the primer sets, each PCR product of approximately 1 kbp is given.

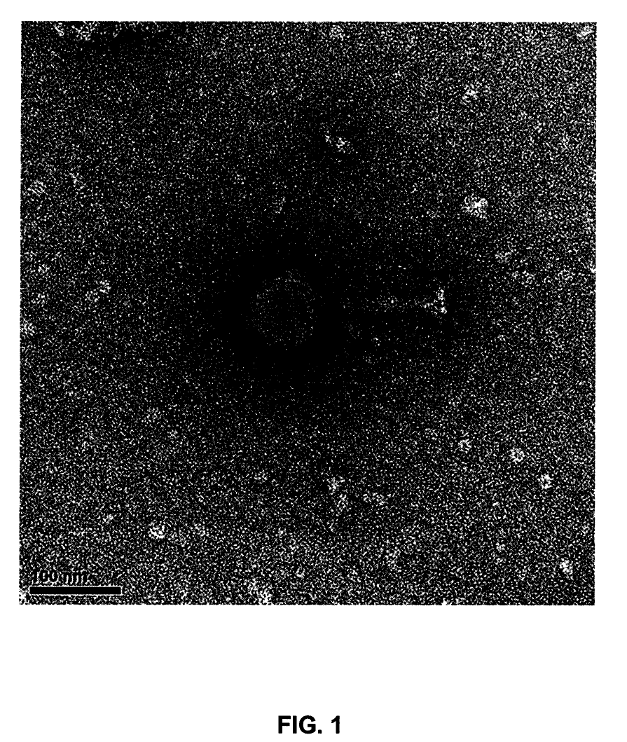

The bacteriophage of the present invention belongs to the morphotype group of the family Myoviridae, characterized by an isometric capsid and a long contractile tail, and preferably the morphology depicted in FIG. 1.

As used herein, the term “nucleic acid molecule” encompasses DNA (gDNA and cDNA) and RNA molecules, and the term nucleotide, as the basic structural unit of nucleic acids, encompasses natural nucleotides and sugar or base-modified analogues thereof.

The bacteriophage of the present invention genetically has major structural proteins with the size of 44-46 kDa, 61-63 kDa, and 79-81 kDa, and preferably the size of approximately 45 kDa, 62 kDa, and 80 kDa.

Further, the bacteriophage of the present invention has one or more biochemical properties of acid-, heat-, and dry-resistance.

More specifically, the bacteriophage of the present invention can stably survive in a wide range of pH environment from pH 3.5 to pH 9.0, and in a high temperature environment from 37° C. to 60° C. In addition, the bacteriophage of the present invention is resistant to dessication to remain viable even after high-temperature drying (e.g., at 60° C. for 120 min). Such properties of acid-, heat-, and drying-resistance allow application of the bacteriophage of the present invention under various temperature and pH conditions upon the production of prophylactic or therapeutic compositions for livestock diseases or human diseases caused by the contaminated livestock.

The present inventors collected sewage samples at chicken slaughterhouses, and isolated the bacteriophage of the present invention that has a specific bactericidal activity against SE, ST, SG and SP and the above characteristics, which was designated as ΦCJ3 and deposited at the Korean Culture Center of Microorganisms (361-221, Yurim B/D, Hongje-1-dong, Seodaemun-gu, Seoul 120-091, Korea) on Dec. 17, 2008 under accession number KCCM10977P.

In accordance with the specific Example of the present invention, the present inventors collected sewage samples at chicken slaughterhouses to isolate bacteriophages that lyse the host cell ST, and they confirmed that the bacteriophages are able to lyse SE, ST, SG and SP. Further, they examined the bacteriophage (ΦCJ3) under electron microscope, and found that it belongs to the morphotype of the family Myoviridae (FIG. 1).

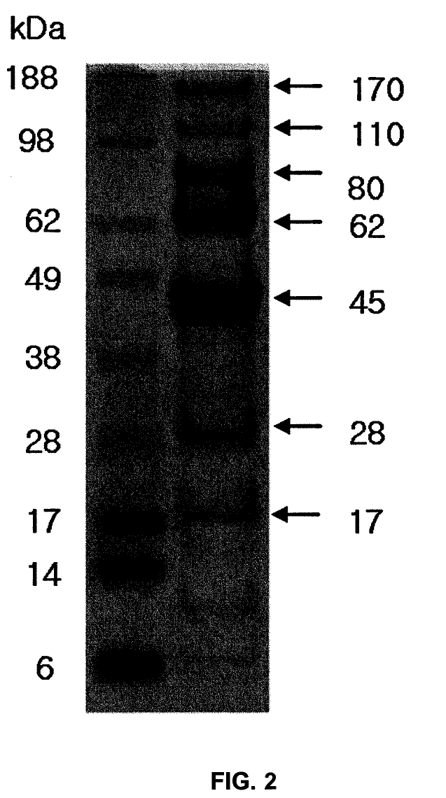

Further, the protein patterns of the bacteriophage ΦCJ3 were also analyzed, resulting in that it has major structural proteins with the size of 45 kDa, 62 kDa, and 80 kDa (FIG. 2).

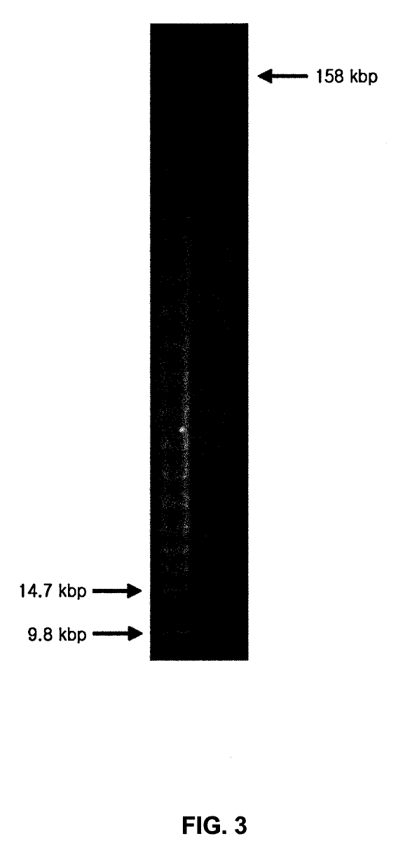

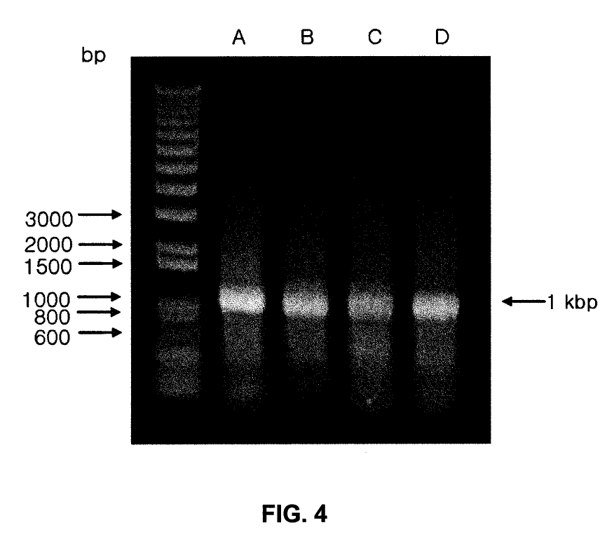

Furthermore, the total genome size of the bacteriophage ΦCJ3 was also analyzed, resulting in that it has a total genome size of approximately 158 kbp (FIG. 3). The results of analyzing its genetic features showed that the bacteriophage includes nucleic acid molecules represented by SEQ ID NOs. 1 to 4 within the total genome. Based on these results, genetic similarity with other species was compared. It was found that the bacteriophage showed very low genetic similarity with the known bacteriophages, indicating that the bacteriophage is a novel bacteriophage (Table 2). More particularly, the ΦCJ3-specific primer sets, namely, SEQ ID NOs. 5 and 6, SEQ ID NOs. 7 and 8, SEQ ID NOs. 9 and 10, and SEQ ID NOs. 11 and 12 were used to perform PCR. Each PCR product was found to have a size of approximately 1 kbp (FIG. 4).

Further, when SE, ST, SG and SP were infected with ΦCJ3, the phage plaques (clear zone on soft agar created by host cell lysis of one bacteriophage) showed the same size and turbidity.

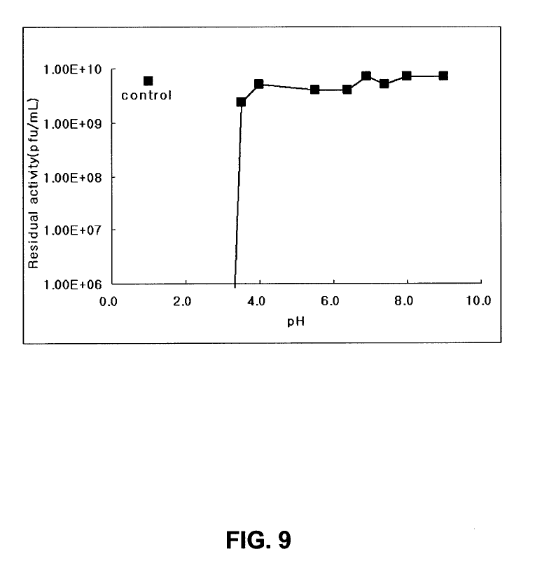

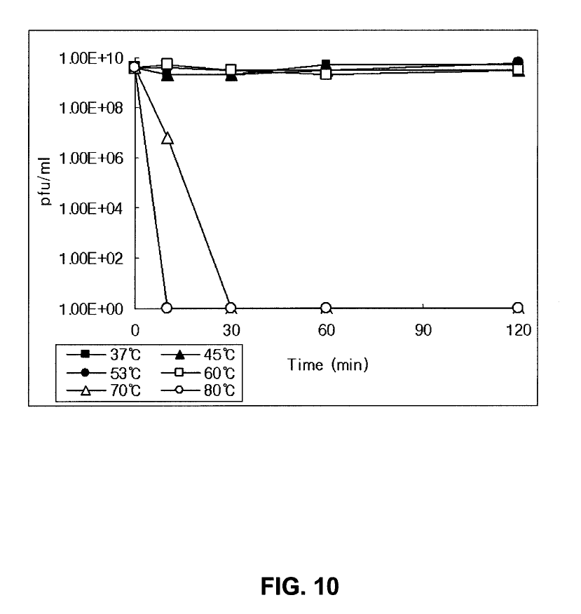

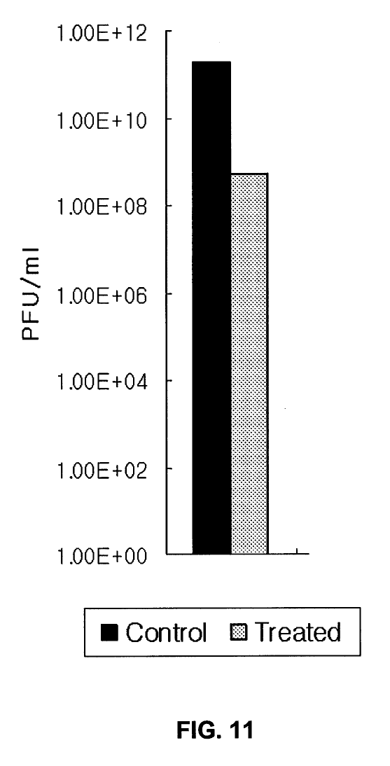

Furthermore, the stability of ΦCJ3 was examined under various temperature and pH conditions, resulting in that ΦCJ3 stably maintains in a wide range of pH environments from pH 3.5 to pH 9.0 (FIG. 9) and in high temperature environments from 37° C. to 60° C. (FIG. 10), and even after high-temperature drying (FIG. 11). These results indicate that the bacteriophage ΦCJ3 of the present invention can be applied to various products for the control of salmonella bacteria.

In accordance with another aspect, the present invention relates to a composition for the prevention or treatment of infectious diseases caused by one or more selected from the group consisting of Salmonella Gallinarum, Salmonella Pullorum, Salmonella Typhimurium, and Salmonella Enteritidis, comprising the bacteriophage as an active ingredient.

Preferably, the infectious diseases caused by Salmonella Enteritidis or Salmonella Typhimurium include salmonellosis and Salmonella food poisoning, the infectious diseases caused by Salmonella Gallinarum include Fowl Typhoid, and the infectious diseases caused by Salmonella Pullorum include Pullorum disease, but are not limited thereto.

The bacteriophage of the present invention has a specific bactericidal activity against Salmonella Gallinarum, Salmonella Pullorum, Salmonella Typhimurium, and Salmonella enteritidis, and thus can be used for the purpose of preventing or treating diseases that are caused by these bacteria. Specifically, in the preferred embodiment, an antibiotic may be included.

As used herein, the term “prevention” means all of the actions in which disease progress is restrained or retarded by the administration of the composition. As used herein, the term “treatment” means all of the actions in which the patient's condition has taken a turn for the better or been modified favorably by the administration of the composition.

The composition of the present invention comprises ΦCJ3 of 5×10² to 5×10¹² pfu/ml, and preferably 1×10⁶ to 1×10¹⁰ pfu/ml.

Preferred examples of infectious diseases, to which the composition of the present invention can be applied, include Fowl Typhoid caused by Salmonella Gallinarum, Pullorum disease caused by Salmonella Pullorum, and salmonellosis or Salmonella food poisoning caused by Salmonella enteritidis or Salmonella Typhimurium, but are not limited thereto.

As used herein, the term “salmonellosis” refers to symptoms caused by Salmonella infection, including fever, headache, diarrhea, and vomiting, namely, diseases caused by bacteria of the genus Salmonella, which is defined two clinical forms—an acute septicemic form that resembles typhoid fever and an acute gastroenteritis, including enteritis, food poisoning, and acute septicemia.

The composition of the present invention may additionally include a pharmaceutically acceptable carrier, and formulated together with the carrier to provide foods, medicines, and feed additives.

As used herein, the term “pharmaceutically acceptable carrier” refers to a carrier or diluent that does not cause significant irritation to an organism and does not abrogate the biological activity and properties of the administered compound. For formulation of the composition into a liquid preparation, a pharmaceutically acceptable carrier which is sterile and biocompatible may be used such as saline, sterile water, Ringer's solution, buffered physiological saline, albumin infusion solution, dextrose solution, maltodextrin solution, glycerol, and ethanol. These materials may be used alone or in any combination thereof. If necessary, other conventional additives may be added such as antioxidants, buffers, bacteriostatic agents, and the like. Further, diluents, dispersants, surfactants, binders and lubricants may be additionally added to the composition to prepare injectable formulations such as aqueous solutions, suspensions, and emulsions, or oral formulations such as pills, capsules, granules, or tablets.

The prophylactic or therapeutic compositions of the present invention may be applied or sprayed to the afflicted area, or administered by oral or parenteral routes. The parenteral administration may include intravenous, intraperitoneal, intramuscular, subcutaneous or topical administration.

The dosage suitable for applying, spraying, or administrating the composition of the present invention will depend upon a variety of factors including formulation method, the mode of administration, the age, weight, sex, condition, and diet of the patient or animal being treated, the time of administration, the route of administration, the rate of excretion, and reaction sensitivity. A physician or veterinarian having ordinary skill in the art can readily determine and prescribe the effective amount of the composition required.

Examples of the oral dosage forms suitable for the composition of the present invention include tablets, troches, lozenges, aqueous or emulsive suspensions, powder or granules, emulsions, hard or soft capsules, syrups, or elixirs. For formulation such as tablets and capsules, useful are a binder such as lactose, saccharose, sorbitol, mannitol, starch, amylopectin, cellulose or gelatin, an excipient such as dicalcium phosphate, a disintegrant such as corn starch or sweet potato starch, a lubricant such as magnesium stearate, calcium stearate, sodium stearylfumarate, or polyethylene glycol wax. For capsules, a liquid carrier such as lipid may be further used in addition to the above-mentioned compounds.

For non-oral administration, the composition of the present invention may be formulated into injections for subcutaneous, intravenous, or intramuscular routes, suppositories, or sprays inhalable via the respiratory tract, such as aerosols. Injection preparations may be obtained by dissolving or suspending the composition of the present invention, together with a stabilizer or a buffer, in water and packaging the solution or suspension in ampules or vial units. For sprays, such as aerosol, a propellant for spraying a water-dispersed concentrate or wetting powder may be used in combination with an additive.

As used herein, the term “antibiotic” means any drug that is applied to animals to kill pathogens, and used herein as a general term for antiseptics, bactericidal agents and antibacterial agents. The animals are mammals including human, and preferably poultry. The bacteriophage of the present invention, unlike the conventional antibiotics, has a high specificity to Salmonella so as to kill the specific pathogens without affecting beneficial bacteria, and does not induce resistance so that its life cycling is comparatively long.

In accordance with one specific embodiment of the present invention, toxicity test on the bacteriophage ΦCJ3 for the prevention of Fowl Typhoid was performed by evaluating its safety and effect on egg production in layer chickens, including its residual amount in chicken flesh and eggs. It was found that there was no difference in the egg production rate between the control and ΦCJ3-treated groups (Table 4), no ΦCJ3 was isolated in the collected eggs (Table 5), and upon treating the SG-infected chickens with ΦCJ3, the ΦCJ3-treated group showed a significantly higher protection rate than the non-treated group (Table 7), indicating its preventive and therapeutic effects.

In accordance with still another aspect, the present invention relates to an animal feed or drinking water, comprising the bacteriophage as an active ingredient.

Feed additive antibiotics used in fishery and livestock industry are used for the purpose of preventing infections, but lead to an increase in resistant strains of bacteria and the residual antibiotics in livestock products may be ingested by humans, contributing to antibiotic resistance in human pathogens and the spread of diseases. In addition, since there are a variety of feed additive antibiotics, the increasing global emergence of multidrug-resistant strain is a serious concern. Therefore, the bacteriophage of the present invention can be used as a feed additive antibiotic that is more eco-friendly and able to solve the above problems.

The bacteriophage of the present invention may be separately prepared as a feed additive, and then added to the animal feed, or directly added to the animal feed. The bacteriophage of the present invention may be contained in the animal feed as a liquid or in a dried form, preferably in a dried powder. The drying process may be performed by air drying, natural drying, spray drying, and freeze-drying, but is not limited thereto. The bacteriophage of the present invention may be added as a powder form in an amount of 0.05 to 10% by weight, preferably 0.1 to 2% by weight, based on the weight of animal feed. The animal feed may also include other conventional additives for the long-term preservation, in addition to the bacteriophage of the present invention.

The feed additive of the present invention can additionally include other non-pathogenic microorganisms. The additional microorganism can be selected from a group consisting of Bacillus subtilis that can produce protease, lipase and invertase, Lactobacillus sp. strain having an ability to decompose organic compounds and physiological activity under anaerobic conditions, filamentous fungi like Aspergillus oryzae (J Animal Sci 43: 910-926, 1976) that increases the weight of domestic animals, enhances milk production and helps digestion and absorptiveness of feeds, and yeast like Saccharomyces cerevisiae (J Anim Sci 56:735-739, 1983).

The feed comprising ΦCJ3 of the present invention may include plant-based feeds, such as grain, nut, food byproduct, seaweed, fiber, drug byproduct, oil, starch, meal, and grain byproduct, and animal-based feeds such as protein, mineral, fat, single cell protein, zooplankton, and food waste, but is not limited thereto.

The feed additive comprising ΦCJ3 of the present invention may include binders, emulsifiers, and preservatives for the prevention of quality deterioration, amino acids, vitamins, enzymes, probiotics, flavorings, non-protein nitrogen, silicates, buffering agents, coloring agents, extracts, and oligosaccharides for the efficiency improvement, and other feed premixtures, but is not limited thereto.

Further, the supply of drinking water mixed with the bacteriophage of the present invention can reduce the number of Salmonella bacteria in the intestine of livestock, thereby obtaining Salmonella-free livestock.

In accordance with still another aspect, the present invention relates to a sanitizer and a cleaner, comprising the bacteriophage as an active ingredient.

In order to remove Salmonella, the sanitizer comprising the bacteriophage as an active ingredient can be used in the poultry barns, slaughterhouses, contaminated areas, and other production facilities, but is not limited thereto.

Further, the cleaner comprising the bacteriophage as an active ingredient can be applied to the contaminated skin, feather, and other contaminated body parts of living animals, in order to remove Salmonella.

In accordance with still another aspect, the present invention relates to a method for preventing or treating infectious diseases caused by one or more Salmonella bacteria selected from the group consisting of Salmonella Enteritidis, Salmonella Typhimurium, Salmonella Gallinarum, and Salmonella Pullorum using the bacteriophage or the composition.

The composition of the present invention may be administered into animals in a pharmaceutical formulation or as a component of the animal feed or in their drinking water, preferably administered by mixing into the animal feed as a feed additive.

The composition of the present invention may be administered in a typical manner via any route such as oral or parenteral routes, in particular, oral, rectal, topical, intravenous, intraperitoneal, intramuscular, intraarterial, transdermal, intranasal, and inhalation routes.

The method for treating the diseases of the present invention includes administration of a pharmaceutically effective amount of the composition of the present invention. It will be obvious to those skilled in the art that the total daily dose should be determined through appropriate medical judgment by a physician. The therapeutically effective amount for patients may vary depending on various factors well known in the medical art, including the kind and degree of the response to be achieved, the patient's condition such as age, body weight, state of health, sex, and diet, time and route of administration, the secretion rate of the composition, the time period of therapy, concrete compositions according to whether other agents are used therewith or not, etc.

Hereinafter, the present invention will be described in more detail with reference to the following Examples. However, these Examples are for illustrative purposes only, and the invention is not intended to be limited by these Examples.

### EXAMPLE 1

### Salmonella Bacteriophage Isolation

### 1-1. Bacteriophage Screening and Single Bacteriophage Isolation

50 ml of sample from chicken slaughterhouse and sewage effluent was transferred to a centrifuge tube, and centrifuged at 4000 rpm for 10 minutes. Then, the supernatant was filtered using a 0.45 μm filter. 18 ml of sample filtrate was mixed with 150 μl of ST shaking culture medium (OD₆₀₀=2) and 2 ml of 10× Luria-Bertani medium (Hereinbelow, designated as LB medium, tryptone 10 g; yeast extract 5 g; NaCl 10 g; final volume to 1 L). The mixture was cultured at 37° C. for 18 hours, and the culture medium was centrifuged at 4000 rpm for 10 minutes. The supernatant was filtered using a 0.2 μm filter. 3 ml of 0.7% agar (w/v) and 150 μl of ST shaking culture medium (OD₆₀₀=2) were mixed, and plated onto LB plate, changed to a solid medium. 10 μl of culture filtrate was spread thereon, and cultured for 18 hours at 37° C. (0.7% agar was used as “top-agar” and the titration of phage lysate was performed on the top-agar, called soft agar overlay method).

The sample culture medium containing the phage lysate was diluted, and mixed with 150 μl of ST shaking culture medium (OD₆₀₀=2), followed by soft agar overlay method, so that single plaques were obtained. Since a single plaque represents one bacteriophage, to isolate single bacteriophages, one plaque was added to 400 μl of SM solution (NaCl, 5.8 g; MgSO₄7H₂O, 2 g; 1 M Tris-Cl (pH 7.5), 50 ml; H₂O, final volume to 1 L), and left for 4 hours at room temperature to isolate single bacteriophages. To purify the bacteriophage in large quantities, 100 μl of supernatant was taken from the single bacteriophage solution, and mixed with 12 ml of 0.7% agar and 500 μl of ST shaking culture medium, followed by soft agar overlay method on LB plate (150 mm diameter). When lysis was completed, 15 ml of SM solution was added to the plate. The plate was gently shaken for 4 hours at room temperature to elute the bacteriophages from the top-agar. The SM solution containing the eluted bacteriophages was recovered, and chloroform was added to a final volume of 1%, mixed well for 10 minutes. The solution was centrifuged at 4000 rpm for 10 minutes. The obtained supernatant was filtered using a 0.2 μm filter, and stored in the refrigerator.

### 1-2. Large-Scale Batches of Bacteriophage

The selected bacteriophages were cultured in large quantities using ST. ST was shaking-cultured, and an aliquot of 1.5×10¹⁰ cfu (colony forming units) was centrifuged at 4000 rpm for 10 minutes, and the pellet was resuspended in 4 ml of SM solution. The bacteriophage of 7.5×10⁷ pfu (plaque forming unit) was inoculated thereto (MOI: multiplicity of infection=0.005), and left at 37° C. for 20 minutes. The solution was inoculated into 150 ml of LB media, and cultured at 37° C. for 5 hours. Chloroform was added to a final volume of 1%, and the culture solution was shaken for 20 minutes. DNase I and RNase A were added to a final concentration of 1 μg/ml, respectively. The solution was left at 37° C. for 30 minutes. NaCl and PEG (polyethylene glycol) were added to a final concentration of 1 M and 10% (w/v), respectively and left at 37° C. for additional 3 hours. The solution was centrifuged at 4° C. and 12000 rpm for 20 minutes to discard the supernatant. The pellet was resuspended in 5 ml of SM solution, and left at room temperature for 20 minutes. 4 ml of chloroform was added thereto and mixed well, followed by centrifugation at 4° C. and 4000 rpm for 20 minutes. The supernatant was filtered using a 0.2 μm filter, and ΦCJ3 was purified by glycerol density gradient ultracentrifugation (density: 40%, 5% glycerol at 35,000 rpm and 4° C. for 1 hour). The purified bacteriophge was designated as bacteriophage ΦCJ3, and resuspended in 300 μl of SM solution, followed by titration. The bacteriophage ΦCJ3 was deposited at the Korean Culture Center of Microorganisms (361-221, Honje 1, Seodaemun, Seoul) on Dec. 17, 2008 under accession number KCCM10977P.

### EXAMPLE 2

### Examination on ΦCJ3 Infection of Salmonella

To examine the lytic activity of the selected bacteriophages on other Salmonella species as well as ST, cross-infection attempts with other Salmonella species were made. As a result, ΦCJ3 did not infect SC (Salmonella enterica Serotype Choleraesuis), SD (Salmonella enterica Serotype Derby), SA (Salmonella enterica subsp. Arizonae), SB (Salmonella enterica subsp. Bongori), but specifically infected SG, SP, ST, and SE (see Example 12). The results are shown in the following Table 1. The bacteriophages ftCJ3 that were produced using SG as a host cell showed the same plaque size and plaque turbidity, and the same protein patterns and genome size as those produced using ST as a host cell.

TABLE 1
ΦCJ3 infection of Salmonella

| Sero | Strain | Plaque | Sero | Strain | Plaque |
|---|---|---|---|---|---|
| type | name | formation | type | name | formation |
| SE | SGSC 2282 | ◯ | SA | ATCC | X |
| | | | | 13314 | |
| ST | ATCC | ◯ | SB | ATCC | X |
| | 14028 | | | 43975 | |
| SG | SGSC 2293 | ◯ | SC | ATCC | X |
| | | | | 10708 | |
| SP | SGSC 2295 | ◯ | SD | ATCC | X |
| | | | | 6960 | |

ATCC: The Global Bioresource Center
SGSC: salmonella genetic stock center

### EXAMPLE 3

### Morphology Examination of Bacteriophage ΦCJ3

The purified ΦCJ3 was diluted in 0.01% gelatin solution, and then fixed in 2.5% glutaraldehyde solution. After the sample was dropped onto a carbon-coated mica plate (ca.2.5×2.5 mm) and adapted for 10 minutes, it was washed with sterile distilled water. Carbon film was mounted on a copper grid, and stained with 4% uranyl acetate for 30-60 seconds, dried, and examined under JEM-1011 transmission electron microscope (80 kV, magnification of ×120,000˜×200,000). As a result, the purified ΦCJ3 had morphological characteristics including an isometric capsid and a long contractile tail, as shown in FIG. 1, indicating that it belongs to the morphotype group of the family Myoviridae.

### EXAMPLE 4

### Protein Pattern Analysis of Bacteriophage ΦCJ3

15 μl of purified ΦCJ3 solution (10¹¹ pfu/ml titer) was treated with 3 μl of 5×SDS sample solution, and heated for 5 minutes. The total protein of ΦCJ3 was run in 4-12% NuPAGE Bis-Tris gel (Invitrogen), and then the gel was stained with Coomassie blue for 1 hour at room temperature. As shown in FIG. 2, the protein patterns showed that 45 kDa, 62 kDa, and 80 kDa bands were observed as major proteins, and 17 kDa, 28 kDa, 110 kDa, and 170 kDa bands were also observed.

### EXAMPLE 5

### Total Genomic DNA Size Analysis of Bacteriophage ΦCJ3

Genomic DNA was isolated from the purified ΦCJ3 by ultracentrifugation. Specifically, to the purified ΦCJ3 culture medium, EDTA (ethylenediaminetetraacetic acid (pH8.0)), proteinase K, and SDS (sodium dodecyl sulfate) were added to a final concentration of 20 mM, 50 μg/ml, and 0.5% (w/v), respectively and left at 50° C. for 1 hour. An equal amount of phenol (pH8.0) was added and mixed well, followed by centrifugation at 12000 rpm and room temperature for 10 minutes. The supernatant was mixed well with an equal amount of PC (phenol:chloroform=1:1), followed by centrifugation at 12000 rpm and room temperature for 10 minutes. The supernatant was mixed well with an equal amount of chloroform, followed by centrifugation at 12000 rpm and room temperature for 10 minutes. Again, to the supernatant, added were 1/10 volume of 3 M sodium acetate and two volumes of cold 95% ethanol, and left at −20° C. for 1 hour. After centrifugation at 0° C. and 12000 rpm for 10 minutes, the supernatant was completely removed, and the DNA pellet was dissolved in 50 μl TE (Tris-EDTA (pH 8.0)). The extracted DNA was diluted 10-fold, and its absorbance was measured at OD₂₆₀. After loading 1 μg of total genomic DNA in 1% PFGE (pulse-field gel electrophoresis) agarose gel, electrophoresis was performed using a BIORAD PFGE system program 7 (size range 25-100 kbp; switch time ramp 0.4-2.0 seconds, linear shape; forward voltage 180 V; reverse voltage 120 V) at room temperature for 20 hours. As shown in FIG. 3, ΦCJ3 had a genomic DNA size of approximately 158 kbp.

### EXAMPLE 6

### Genetic Analysis of Bacteriophage ΦCJ3

To analyze genetic features of the purified ΦCJ3, 5 μg of genomic DNA of ΦCJ3 was treated with the restriction enzymes, EcoR V and Sca I. The vector, pBluescript SK+ (Promega) was digested with EcoR V, and treated with CIP (calf intestinal alkaline phosphatase). The digested genomic DNA and vector were mixed in a ratio of 3:1, and ligated at 16° C. for 5 hours. The ligation product was transformed into E. coli DH5a. The transformed cells were plated on LB plate containing ampicillin and X-gal (5-bromo-4-chloro-3-indolyl-beta-D-galactopyranoside) for blue/white selection, so as to select four colonies. The selected colony was shaking-cultured in a culture medium containing ampicillin for 16 hours. Then, plasmids were extracted using a plasmid purification kit (Promega).

The cloning of the plasmids was confirmed by PCR using a primer set of M13 forward and M13 reverse, and insert fragments of 1 kbp or more were selected, and their base sequence was analyzed using the primer set of M13 forward and M13 reverse. As shown in SEQ ID NOs. 1 and 4, all have the size of 1 kbp. Sequence similarity was analyzed using a NCBI blastx program, and the results are shown in Table 2.

As shown in Table 2, ΦCJ3 showed no sequence similarity with other proteins in the upstream region of SEQ ID NO. 1, and about 40% sequence similarity with the single-stranded DNA binding protein of synechococcus phage in the downstream region of the sequence in a backward direction. The upstream region of SEQ ID NO. 2 showed about 32% sequence similarity with the sliding clamp protein of synechococcus phage in a backward direction. The downstream region also showed about 32% sequence similarity with the UvsW RNA-DNA and DNA-DNA helicase ATPase of ecterobacteria phage Phil in a backward direction. The sequence of SEQ ID NO. 3 showed no sequence similarity with the proteins of bacteriophage. The downstream region of SEQ ID NO. 3 showed about 29% sequence similarity with ATP-dependent DNA helicase RecG of psychroflexus torques, and the upstream region of SEQ ID NO. 3 showed about 38% sequence similarity with the conserved protein of leishmania major. The upstream region of SEQ ID NO. 4 showed about 46% sequence similarity with the UvsX RecA-like recombination protein of enterobacteria phage in a backward direction.

Further, SEQ ID NOs. 2 and 3 of ΦCJ3 showed high e-value, and the sequence of SEQ ID NO. 3 showed no sequence similarity with the proteins of bacteriophage. Furthermore, as a result of the base sequence analysis of SEQ ID NOs. 1 to 4 by NCBI blastn program, no sequence similarity was observed. These results indicate that ΦCJ3 is a novel bacteriophage.

TABLE 2
Sequence similarity comparison of ΦCJ3 with other bacteriophages

| | | | Accession | Subject | Query | | E |
|---|---|---|---|---|---|---|---|
| | Organism | Protein | number | location | location | Identities | value |
| 1 | Synechococcus | single- | NC_008296.2 | 68-247 | 995-429 | 77/192 | 2e−30 |
| | phage | stranded | | | | (40%) | |
| | syn9 | DNA | | | | | |
| | | binding | | | | | |
| | | protein | | | | | |
| | Synechococcus | ssDNA | NC_006820.1 | 63-242 | 995-423 | 79/202 | 3e−29 |
| | phage S- | binding | | | | (39%) | |
| | PM2 | protein | | | | | |
| | | gp32 | | | | | |
| 2 | Enterobacteria | UvsW | NC_009821.1 | 443-500 | 998-855 | 19/58 | 1.9 |
| | phage | RNA-DNA | | | | (32%) | |
| | Phi1 | and | | | | | |
| | | DNA-DNA | | | | | |
| | | helicase | | | | | |
| | | ATPase | | | | | |
| | Synechococcus | sliding | NC_008296.2 | 4-45 | 126-1 | 19/58 | 4.5 |
| | phage | clamp | | | | (32%) | |
| | syn9 | | | | | | |
| 3 | Psychroflexus | ATP- | NZ_AAPR01000001.1 | 12-136 | 541-873 | 38/129 | 0.045 |
| | torquis | dependent | | | | (29%) | |
| | ATCC | DNA | | | | | |
| | 700755 | helicase | | | | | |
| | | RecG | | | | | |
| | Leishmania | hypothetical | XM_001681761.1 | 58-115 | 121-297 | 23/59 | 0.22 |
| | major | protein, | | | | (38%) | |
| | | conserved | | | | | |
| 4 | Enterobacteria | UvsX | NC_004928.1 | 34-225 | 578-3 | 89/192 | 2e−41 |
| | phage | RecA- | | | | (46%) | |
| | RB69 | like | | | | | |
| | | recombination | | | | | |
| | | protein | | | | | |
| | Enterobacteria | UvsX | NC_010105.1 | 34-225 | 578-3 | 87/192 | 3e−41 |
| | phage | RecA- | | | | (45%) | |
| | JS98 | like | | | | | |
| | | recombination | | | | | |
| | | protein | | | | | |

### EXAMPLE 7

### Construction of ΦCJ3-specific primer sequence

To identify ΦCJ3, ΦCJ3-specific primers were constructed on the basis of SEQ ID NOs. 1 and 4. PCR was performed using each primer set of SEQ ID NOs. 5 and 6, SEQ ID NOs. 7 and 8, SEQ ID NOs. 9 and 10, and SEQ ID NOs. 11 and 12. 0.1 μg of genomic DNA of bacteriophage and 0.5 pmol of primer were added to pre-mix (Bioneer), and the final volume was adjusted to 20 μl. PCR was performed with 30 cycles of denaturation; 94° C. 30 sec, annealing; 60° C. 30 sec, and polymerization; 72° C., 1 min. When SEQ ID NOs. 5 and 6, SEQ ID NOs. 7 and 8, SEQ ID NOs. 9 and 10, and SEQ ID NOs. 11 and 12 were used as primer set, all PCR products of approximately 1 kbp were obtained. The results are shown in FIG. 4.

### EXAMPLE 8

### Test on Infection Efficiency of Bacteriophage

To test the infection efficiency of the bacteriophage ΦCJ3, a one-step growth experiment was performed.

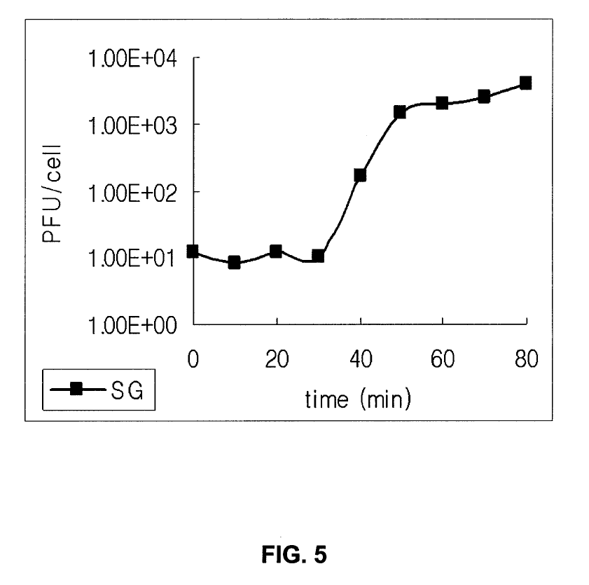

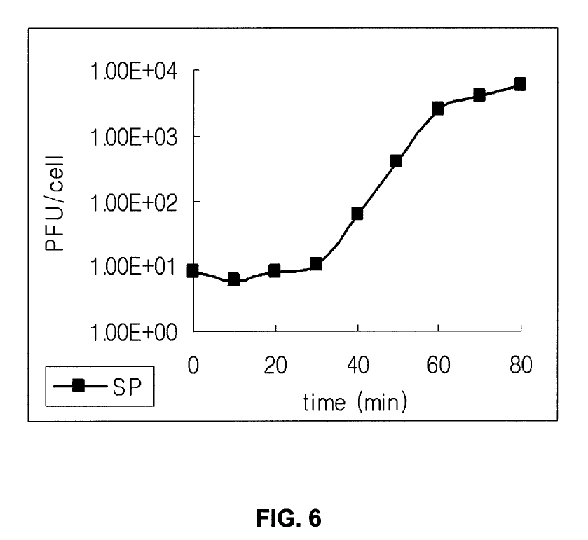

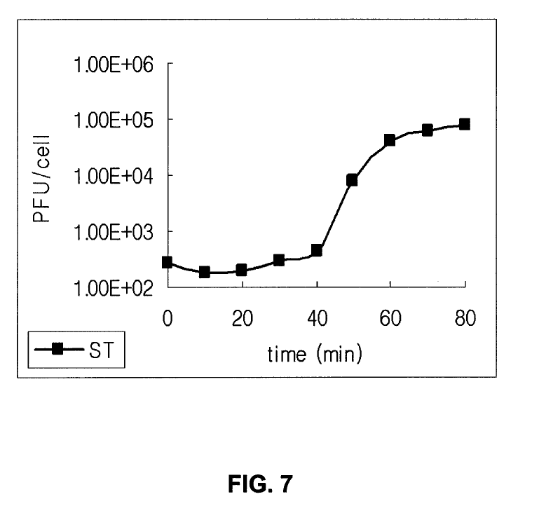

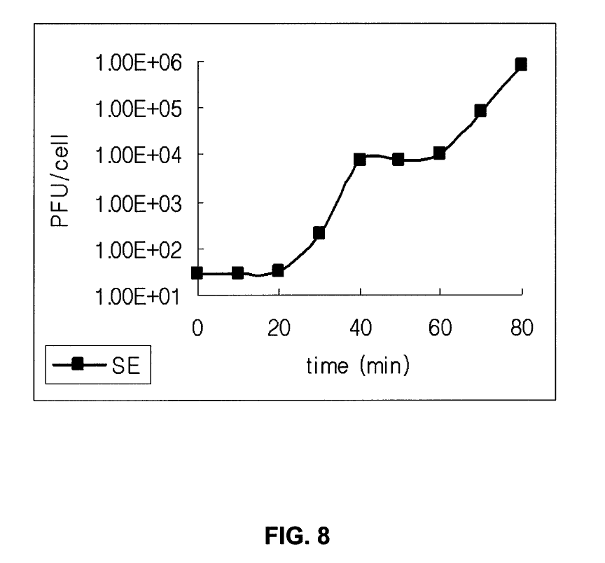

50 ml of SG culture medium (OD₆₀₀=0.5) was centrifuged at 4000 rpm for 10 minutes, and resuspended in 25 ml of fresh LB medium. The purified bacteriophage (MOI=0.0005) was inoculated thereto, and left for 5 minutes. The reaction solution was centrifuged at 4000 rpm for 10 minutes, and the cell pellet was resuspended in fresh LB medium. While the cells were cultured at 37° C., two samples of cell culture medium were collected every 10 minutes, and centrifuged at 12000 rpm for 3 minutes. The obtained supernatant was serially diluted, and 10 μl of each diluted sample was cultured at 37° C. for 18 hours by soft agar overlay method, and the titration of phage lysates was performed. Thus, the same experiment was performed on SP, ST, and SE. The result of one-step growth experiment on SG, SP, ST, and SE showed the burst size of 2×10² or more. The results are shown in FIGS. 5 to 8.

### EXAMPLE 9

### pH Stability Test on Bacteriophage

To test the stability of ΦCJ3 in a low-pH environment like the livestock stomach, the stability test was performed in a wide range of pH (pH 2.1, 2.5, 3.0, 3.5, 4.0, 5.5, 6.4, 6.9, 7.4, 8.2, 9.0). Various pH solutions (Sodium acetate buffer (pH 2.1, pH 4.0, pH 5.5, pH 6.4)), Sodium citrate buffer (pH 2.5, pH 3.0, pH 3.5), Sodium phosphate buffer (pH 6.9, pH 7.4), Tris-HCl (pH 8.2, pH 9.0)) were prepared at a concentration of 2 M. 100 μl of pH solution was mixed with an equal amount of bacteriophage solution (1.0×10¹⁰ pfu/ml) to the concentration of each pH solution to 1 M, and left at room temperature for 1 hour. The reaction solution was serially diluted, and 10 μl of each diluted sample was cultured at 37° C. for 18 hours by soft agar overlay method, and the titration of phage lysates was performed. Changes in the titers according to pH difference were compared to examine the relative stability. The results showed that the bacteriophage did not lose its activity and maintained stability until pH 3.5. However, it lost its activity at pH 3.0 or lower. The results are shown in FIG. 9.

### EXAMPLE 10

### Heat Stability Test on Bacteriophage

To test stability of bacteriophage to heat generated during formulation process when used as a feed additive, the following experiment was performed. 200 μl of ΦCJ3 solution (1.0×10¹⁰ pfu/ml) was left at 37° C., 45° C., 53° C., 60° C., 70° C., and 80° C., for 0 min, 10 min, 30 min, 60 min, and 120 min, respectively. The solution was serially diluted, and 10 μl of each diluted sample was cultured at 37° C. for 18 hours by soft agar overlay method, and the titration of phage lysates was performed. Changes in the titers according to temperature and exposure time were compared to examine the relative stability. The results showed that the bacteriophage did not lose its activity at 60° C. until the exposure time reached 2 hours. However, the bacteriophage lost its activity at 70° C. or higher. The results are shown in FIG. 10.

### EXAMPLE 11

### Dry Stability Test on Bacteriophage

To test stability of bacteriophage under the dry condition used during the formulation process for feed additive, the following experiment was performed. On the basis of the results of heat stability test, the experiment was performed under high-temperature drying conditions (60° C. for 120 min). 200 μl of ΦCJ3 solution (1.0×10¹¹ pfu/ml) was dried using a Speed vacuum (Speed—Vacuum Concentrator 5301, Eppendorf). The obtained pellet was completely resuspended in an equal amount of SM solution at 4° C. for one day. The solution was serially diluted, and 10 μl of each diluted sample was cultured at 37° C. for 18 hours by soft agar overlay method, and the titration of phage lysates was performed. Changes in the titers before and after drying were compared to examine the relative stability. The results showed that its activity was decreased to 5×10³. The results are shown in FIG. 11.

### EXAMPLE 12

### Examination on Bacteriophage Infection of Wild-Type Strain

The lytic activity of bacteriophage 003 was tested for the Korean wild-type SE (38 strains), ST (22 strains), SG (56 strains) and SP (19 strains), isolated by Laboratory of Avian Diseases, College of Veterinary Medicine, Seoul National University, and National Veterinary Research and Quarantine Service and the Korea Centers for Disease Control and Prevention, in addition to SG (SG SGSC2293), SP(SP SGSC2295), ST (ST ATCC14028), and SE (SE SCSG 2282) used in the present invention. 150 d of each strain shaking culture medium (OD₆₀₀=2) was mixed, and 10 μl of ΦCJ3 solution (10¹⁰ pfu/ml) was cultured at 37° C. for 18 hours by soft agar overlay method, and the plaque formation was examined. It was found that the bacteriophage ΦCJ3 showed lytic activity of 95%, 58%, 100% and 81% on the wild type SE, ST, SG, and SP, respectively. The results are shown in the following Table 3.

TABLE 3
Lytic activity on Korean wild-type SG, SP, ST, and SE

| Sero | Strain | Plaque | Sero | | Plaque |
|---|---|---|---|---|---|
| type | name | formation | type | Strain name | formation |
| SG | SNU SG1 | ◯ | ST | SNU ST1 | X |
| | SNU SG2 | ◯ | | SNU ST2 | ◯ |
| | SNU SG3 | ◯ | | SNU ST3 | ◯ |
| | SNU SG4 | ◯ | | SNU ST4 | ◯ |
| | SNU SG5 | ◯ | | SNU ST7 | ◯ |
| | SNU SG6 | ◯ | | SNU ST8 | ◯ |
| | SNU SG7 | ◯ | | SNU ST11 | ◯ |
| | SNU SG8 | ◯ | | SNU ST12 | ◯ |
| | SNU SG9 | ◯ | | SNU ST13 | X |
| | SNU SG10 | ◯ | | SNU ST14 | X |
| | SNU SG11 | ◯ | | SNU ST17 | ◯ |
| | SNU SG12 | ◯ | | SNU ST18 | X |
| | SNU SG13 | ◯ | | SNU ST19 | X |
| | SNU SG14 | ◯ | | SNU ST20 | X |
| | SNU SG15 | ◯ | | SNU ST25 | X |
| | SNU SG16 | ◯ | | SNU ST26 | X |
| | SNU SG17 | ◯ | | SNU ST37 | ◯ |
| | SNU SG18 | ◯ | | SNU ST38 | ◯ |
| | SNU SG19 | ◯ | | SNU ST41 | ◯ |
| | SNU SG20 | ◯ | | SNU ST42 | ◯ |
| | SNU SG21 | ◯ | | ATCC UK1 | ◯ |
| | SNU SG22 | ◯ | | ATCC 14028S | ◯ |
| | SNU SG23 | ◯ | | SGSC STM1412 | ◯ |
| | SNU SG24 | ◯ | | SGSC STM260 | ◯ |
| | SNU SG25 | ◯ | | SGSC STM | X |
| | | | | SA2197 | |
| | SNU SG26 | ◯ | SE | SGSC SE2282 | ◯ |
| | SNU SG27 | ◯ | | SGSC SE2377 | ◯ |
| | SNU SG28 | ◯ | | PT4 S1400194 | X |
| | SNU SG30 | ◯ | | PT4 LA52 | ◯ |
| | SNU SG31 | ◯ | | NVRQS SE004 | ◯ |
| | SNU SG32 | ◯ | | NVRQS SE005 | ◯ |
| | SNU SG33 | ◯ | | KCDC SE008 | ◯ |
| | SNU SG34 | ◯ | | KCDC SE009 | ◯ |
| | SNU SG36 | ◯ | | KCDC SE010 | ◯ |
| | SNU SG37 | ◯ | | KCDC SE011 | X |
| | SNU SG38 | ◯ | | KCDC SE012 | ◯ |
| | SNU SG39 | ◯ | | KCDC SE013 | ◯ |
| | SNU SG40 | ◯ | | KCDC SE014 | ◯ |
| | SNU SG41 | ◯ | | KCDC SE015 | ◯ |
| | SNU SG42 | ◯ | | KCDC SE018 | ◯ |
| | SNU SG43 | ◯ | | KCDC SE019 | ◯ |
| | SNU SG44 | ◯ | | KCDC SE020 | ◯ |
| | SNU SG45 | ◯ | | KCDC SE021 | ◯ |
| | SNU SG46 | ◯ | | KCDC SE024 | ◯ |
| | SNU SG47 | ◯ | | KCDC SE025 | ◯ |
| | SNU SG48 | ◯ | | KCDC SE026 | ◯ |
| | SNU SG49 | ◯ | | KCDC SE027 | ◯ |
| | SNU SG50 | ◯ | | KCDC SE028 | ◯ |
| | SGSC | ◯ | | KCDC SE029 | ◯ |
| | SG9184 | | | | |
| | SGSC | ◯ | | KCDC SE030 | ◯ |
| | SG2292 | | | | |
| | SGSC | ◯ | | KCDC SE031 | ◯ |
| | SG2293 | | | | |
| | SGSC | ◯ | | KCDC SE032 | ◯ |
| | SG2744 | | | | |
| | SGSC | ◯ | | KCDC SE033 | ◯ |
| | SG2796 | | | | |
| SP | SNU SP1 | ◯ | | KCDC SE034 | ◯ |
| | SNU SP4 | ◯ | | KCDC SE035 | ◯ |
| | SNU SP5 | ◯ | | KCDC SE036 | ◯ |
| | SNU SP8 | ◯ | | KCDC SE037 | ◯ |
| | SNU SP11 | ◯ | | KCDC SE039 | ◯ |
| | SGSC | ◯ | | KCDC SE040 | ◯ |
| | SP2294 | | | | |
| | SGSC | ◯ | | KCDC SE041 | ◯ |
| | SP2295 | | | | |
| | SGSC | X | | KCDC SE042 | ◯ |
| | SP2737 | | | | |
| | SGSC | X | | KCDC SE043 | ◯ |
| | SP2739 | | | | |
| | SGSC | ◯ | | KCDC SE044 | ◯ |
| | SP2742 | | | | |
| | SGSC | ◯ | | KCDC SE045 | ◯ |
| | SP2743 | | | | |
| | SGSC | ◯ | SC | ATCC SC10708 | X |
| | SP2745 | | | | |
| | SGSC | ◯ | | ATCC SC2929 | X |
| | SP2751 | | | | |
| | SGSC | X | SD | ATCC SD6960 | X |
| | SP4663 | | | | |
| | SGSC | ◯ | | ATCC SD2466 | ◯ |
| | SP4664 | | | | |
| | SGSC | ◯ | | ATCC SD2467 | X |
| | SP4665 | | | | |
| | SGSC | ◯ | | ATCC SD2468 | ◯ |
| | SP4666 | | | | |
| | SGSC | X | SA | ATCC SA13314 | X |
| | SP4667 | | | | |
| | SGSC | ◯ | SB | ATCC SB43975 | X |
| | SA1684 | | | | |

SNU: Laboratory of Avian Diseases, College of Veterinary Medicine, Seoul National University
SGSC: salmonella genetic stock center

### EXAMPLE 13

### Toxicity test on Bacteriophage

Toxicity test on the bacteriophage ΦCJ3 for the prevention of Fowl Typhoid was performed by evaluation of its safety, residual amount, and eggs in layer chickens. The layer chickens are divided into three groups to perform a pathogenicity test and egg test, and to examine the presence of clinical signs and phage content in the cecal feces.

For the pathogenicity test, 13 brown layer chickens were divided into a 1CJ3-treated group with 8 layers and a control group with 5 layers. The ΦCJ3-treated group was fed with a mixture of feed and ΦCJ3 (10⁸ pfu or more per feed (g)) and the control group was fed with feed only, and egg production rate and clinical signs were examined for 3 weeks after phage treatment. As shown in the following Table 4, the ΦCJ3-treated group and the control group showed about 50% and 50% egg production rates, respectively. In addition, when clinical signs were examined after phage treatment, respiratory and digestive lesions were not observed for 24 days after ΦCJ3 treatment, and abnormal activity was not observed. The results indicate that ΦCJ3 treatment does not generate adverse effects.

For the egg test, 10 eggs were collected on day 3, 6, and 9 after ΦCJ3 treatment, and the egg surface was washed with 70% ethanol and broken out. Egg yolk and egg white were mixed, and 5 ml of the mixture was diluted with 45 ml of PBS by 10⁻¹, 10⁻², and 10⁻³. 10⁶ cfu of SNUSG0197 was added to 25 ml of each diluted solution, and incubated at 37° C. for 3 hours, and cells were isolated by centrifugation. 500 μl of supernatant and 100 μl of SNUSG0197 (10⁹ cfu/ml) were mixed with each other, and plated on a tryptic soy agar plate by top-agar overlay technique. After incubation at 37° C. for 18 hrs, the number of plaques was counted to calculate the number of phage per 1 ml of egg. As shown in the following Table 5, no ΦCJ3 was found in 26 eggs that were collected on days 3, 6, and 9.

Next, the presence of clinical signs and ΦCJ3 content in the cecal feces were examined after ΦCJ3 treatment. At 3 weeks after ΦCJ3 treatment, the test layer chickens were euthanatized, and autopsy was performed to examine gross lesions in the liver, spleen, kidney and ovary. The liver sample was aseptically collected with sterile cotton swab, and plated on a MacConkey agar plate to examine the presence of Salmonella Gallinarum. The cecal feces were also collected to measure the ΦCJ3 content in the individual chickens. Briefly, 1 g of cecal feces was suspended in 9 ml of PBS, and centrifuged at 15000 g for 30 min. 1 ml of supernatant was diluted with PBS by 10⁻¹ to 10⁻⁴, and 500 μl of the dilution and 100 μl of SG0197 (10⁹ cfu/ml) were mixed with each other, and plated on a 10× tryptic soy agar plate by top-agar overlay technique. After incubation at 37° C. for 18 hrs, the number of plaques was counted to calculate the number of phage per cecal feces (g), taking into account the serial dilution.

As a result, no abnormal clinical signs were observed during the examination period, and about 3.7×10⁴ pfu of ΦCJ3 per cecal feces (g) was measured, indicating survival of the bacteriophage in the intestine after passing through the stomach.

Bacteriophage distribution in the organs was examined. Briefly, 10 SPF chicks (11 day-old) were divided into two groups with each 5 chicks. For 3 days, the treated group was fed with feed supplemented with 10⁸ pfu of ΦCJ3 (per g), and the control group was fed with feed only. The chicks were sacrificed to collect the liver, kidney and cecal feces, and the presence of ΦCJ3 was examined. Each of the collected liver, kidney and cecal feces was emulsified with an equal volume of PBS. 1 ml of the liver and the whole quantity of the kidney and cecal feces were transferred into 1.5 ml tubes, followed by centrifugation at 15,000 rpm for 15 min 1 ml of supernatant was diluted with PBS by 10⁻¹ to 10⁻⁴, and 500 μl of the dilution and 100 μl of SG0197 (10⁹ cfu/ml) were mixed with each other, and plated on a 10× tryptic soy agar plate by top-agar overlay technique. After incubation at 37° C. for 18 hrs, the number of plaques was counted to calculate the number of bacteriophage per cecal feces (g), taking into account the serial dilution. As shown in the following Table 6, ΦCJ3 was not observed in the liver and kidney, but was observed in the cecal feces.

TABLE 4
Average egg production rate on ΦCJ3 treatment

| | |
|---|---|
| ΦCJ3 | Control |

| | |
|---|---|
| Day 1 | Feeding day |

| | | |
|---|---|---|
| Day 2 | | |
| Day 3 | 10 | 5 |
| Day 6 | 13 | 7 |
| Day 7 | 4 | 3 |
| Day 8 | 8 | 4 |
| Day 9 | 3 | 1 |
| Day 10 | 2 | 3 |
| Day 13 | 13 | 8 |
| Day 14 | | |
| Day 15 | 8 | 7 |
| Day 16 | | |
| Day 17 | 8 | 6 |
| Day 20 | 9 | 7 |
| Day 21 | | |
| Day 22 | 9 | 4 |
| Day 23 | | |
| Day 24 | 9 | 3 |
| Egg | 50.0% | 50.8% |
| production | | |
| rate | | |

TABLE 5
Isolation frequency of ΦCJ3

| Collection | | |
|---|---|---|
| day | ΦCJ3 | Control |
| Day 3 | 0/10 | 0/5 |
| Day 6 | 0/13 | 0/7 |
| Day 9 | 0/3 | 0/1 |
| Total | 0/26 | 0/13 |

TABLE 6
Presence of phages in organs of ΦCJ3-treated group

| | ΦCJ3-treated group | | | Control group | | |
|---|---|---|---|---|---|---|
| Test | | | cecal | | | cecal |
| chicken | liver | kidney | feces | liver | kidney | feces |
| 1 | − | − | + | − | − | − |
| 2 | − | − | + | − | − | − |
| 3 | − | − | + | − | − | − |
| 4 | − | − | + | − | − | − |
| 5 | − | − | + | − | − | − |

### EXAMPLE 14

### Efficacy test on Bacteriophage

In order to evaluate the efficacy of ΦCJ3 on the prevention and treatment of SG, efficacy test was performed in chickens.

20 brown layers (1-day-old) were divided into 10 test groups with 10 layers ΦCJ3 treated group+non-treated challenged group 1). For 1 week, the test chicks were fed with feed supplemented with 10⁷ pfu of ΦCJ3 (per g) and drinking water supplemented with 10⁷ pfu of ΦCJ3 (per ml). At 1 week, 10⁶ cfu of SG0197 (per chick) and 10⁷ pfu (MOI=10) of phage was mixed with 500 μl of TSB, and left in ice for 1 hour or less, followed by oral administration. The mortality rate was examined for 2 weeks. The surviving chicks were sacrificed and subjected to autopsy and examined for gross lesions, and the bacteria were isolated. As shown in the following Table 7, it was found that the ΦCJ3-treated group showed a significantly higher protection rate (P<0.05) than the non-treated group.

TABLE 7
Efficacy test of ΦCJ3 in chickens

| | | Non-treated |
|---|---|---|
| | ΦCJ3-treated | challenged |
| | challenged group | group |
| Survival | 9 | 3 |
| Mortality rate | 10% | 70% |
| Clinical signs | 1/9 | 1/3 |
| SG reisolation | 1/9 | 0/3 |
| Protection | 80% | 20% |
| rate | | |

The novel bacteriophage of the present invention has a specific bactericidal activity against one or more Salmonella bacteria selected from the group consisting of Salmonella Enteritidis (SE), Salmonella Typhimurium (ST), Salmonella Gallinarum (SG), and Salmonella Pullorum (SP) without affecting beneficial bacteria, and excellent acid-, heat- and dry-resistance, and thus can be widely used in therapeutic agents, animal feeds or drinking water, cleaners and sanitizers for the purpose of preventing and treating the infectious diseases caused by Salmonella Enteritidis, Salmonella Typhimurium, Salmonella Gallinarum or Salmonella Pullorum including salmonellosis, Salmonella food poisoning, Fowl Typhoid, and Pullorum disease.

All documents, articles, publications, patents, and patent applications mentioned in this specification are herein incorporated by reference to the same extend as if each individual publication or patent application was specifically and individually indicated to be incorporated by reference.

| | |
|---|---|
| <110> | CJ Cheiljedang Corporation |
| <120> | Novel bacteriophage and antibacterial composition comprising |
| | the same |
| <130> | OPA09112 |
| <160> | 12 |
| <170> | KopatentIn 1.71 |
| <210> | 1 |
| <211> | 1000 |
| <212> | DNA |
| <213> | Bacteriophage KCCM 10977P |
| <400> | 1 |

| | |
|---|---|
| ccctgctggc ttataagcaa ctcgatcagc ttgtaaacaa tctggctggt atcagcttgg | 60 |
| ctgatttcga aaggctctct atcggcatcc agagggatat attaggcaat gataacctga | 120 |
| cggcgtctga gaagagtagt ctgttgggat tgttacagga tgttgtcaaa aactaaaaag | 180 |
| cccccgaagg ggctttagtg aaattagtct tgcttcagga actgctcgaa ctcatcaatg | 240 |
| gaagccgtct gtttcgcatc ggcaccacca ttattggctg gaacagattg ctgtgcatta | 300 |
| gaaggctgag attgttgttg gtttagactt tcctgcgctg ttgggcgctg gggttcctga | 360 |
| gactgggtag gcgcatgtgc catagtagaa gcaccacctt caaccagagg ctgattatca | 420 |
| gggatggcca gaactttgcg caaacgtttt tccagatctt cgtacgattt gaagttggcc | 480 |
| ggattaaaga actcaaacag actgtgttct ttttcccaga tctcttcaat gtattcgtct | 540 |
| gtccccaaag gtgccggagt atcccacttc acattggtga agttggccac cagacctttc | 600 |
| cagttgccga actctttctc ttcgccaaag aggttcagaa tcaggttcgc gccttcccac | 660 |
| atatcgaacg ggtcgaattt agggtcagtt gagaacttag gattctgagc cgaatccagg | 720 |
| attttcttga cggcattacc gaactcaagc aagaagacct tgccgttgtt ttccggattg | 780 |
| ttgccatctt tgatcaccag gatgttggcg tagtatttgg tgtccggcag acgttttttg | 840 |
| agaactgttt tcagcttttc atcattcgtt tctttctgtt gtgcccacag aggacggtca | 900 |
| tggtcacgaa caggatcatc gttaccgaaa gtctgaggag agttttcgat ataccaacca | 960 |
| ccagcaccct ggaatgcgtg tttcatgatc atggcacacg | 1000 |

| | |
|---|---|
| <210> | 2 |
| <211> | 1000 |
| <212> | DNA |
| <213> | Bacteriophage KCCM 10977P |
| <400> | 2 |

| | |
|---|---|
| aatgccggca atagcgataa ctgtactgga atcgttaaca gtgcgcaact ttttaccagg | 60 |
| tgccagaacg atagaggggc agatggtttc aaagttagcc agcagttgca aagtgcgttc | 120 |
| ggagagagtg atctcttgca ttagttgtat cctcaaaata tagtggggct caagtcatat | 180 |
| ttgacgcaaa ctagtatcgc gtgtttgtag ttatagaaca agtgataaat tgccctacgc | 240 |
| gcgataaata aatgcctgac ggcatttata atattctgtt ttaataaaac ctttctttat | 300 |
| cagtctactc gcttcgctcg tgataatact cgttgctcgc aaagctcaca actcgtatat | 360 |
| tacgcacgga ttgttcaaca agaaagcgat ttttattcaa caagtaaaat attttatttg | 420 |
| gtctaaacag cgcatgacat tattatgtag ccaagtttgc taacacgtga gaaataacat | 480 |
| atgaagcaat ttgttggttt atacgcagta ggggaagacc aagaagcaat tctttccata | 540 |
| gcagaacaac gttcgtcatt aaaaggcgtt tatttacaaa gccttttccg tacatcgggg | 600 |
| tttattgtgt caccgatgtt ggtgatacca ttactcccaa ataacaaagg tctgtatgtt | 660 |
| ggcattattc aacaaggcca ggcgcgggaa gtgaaagttg ttccactgct ggcatctaat | 720 |
| gaagaattgt tttctcagat tcttgagccg aaagtactac aacaatgtat cggcacgatc | 780 |
| gactgtttat ttggttccaa caaagaaggc gaggcaaccc ccgcctatgt gaatcaagat | 840 |
| atttgaaatg gttagagcgc cacttttttc attttaacag ggtggcgctc cataagataa | 900 |
| aatttatatc tctcatgaga atgcctgaga gcgtggttgt aggaaccgtt gtagcgcagg | 960 |
| ttgtctacca ggtcccagat tcgcgcaaca tccttagagg | 1000 |

| | |
|---|---|
| <210> | 3 |
| <211> | 1000 |
| <212> | DNA |
| <213> | Bacteriophage KCCM 10977P |
| <400> | 3 |

| | |
|---|---|
| aattatgcaa atggccagca gggcatcaat ggcagcaaac tccgtcaggc catctggctg | 60 |
| atggttgagc acctcaaggc cggaggctcc cctgacatca tccatggcac tgtcgttggt | 120 |
| agtccgcagt cccctatggc gacagcagtc tctcggcact tcggtggcca cacaaccact | 180 |
| gtactcggcg cgaccaaacc cacgacatgt atgaaccacg acatggtggc aatgtcggcc | 240 |
| tggtttggta gtatattcaa cttcgtcggc tcgggataca acagcaccat ccagccgcgg | 300 |
| tgtaagaagt tgatcgaaca acagaatcca aaggcgtatt atctggagta tggtatcacc | 360 |
| ctcgaccata cggcccactc ccctgagcgc attgctgggt tccatatgct gggcggggag | 420 |
| caggttgcca acatccctga ccatattacc gatctaatta tccctgctgg ctcctgcaac | 480 |
| tcatgtacaa gcatcctgac cggactggca atgcatccga aaccaaatct gaagaatgtc | 540 |
| tatctgatcg ggattggacc aaaccgatta gatttcattg aaagtcgttt gcgcattatc | 600 |
| ggtaagcaag caaacctccc tcacataacc gatttcactc gtcgctatca cgacaaccca | 660 |
| gattatgtgt atggtaagaa ggatcttcag catgcctcta agagcgtttc gctggctggc | 720 |
| ctcctaagtg gtatcaggca gaaggacgag ccagaggtaa cgcttcctcg ctttgaggta | 780 |
| caccattggg atcttcatac cactaattgg gttcgttaca acgacctgat ggactaccag | 840 |
| tggggtgata tcgaactgca cccgcgctat gaaggcaagg tcatgacctg gatccagcag | 900 |
| cacaagccag agatgctgaa cgagaacact ctgttctgga tcgttggtag taagccatat | 960 |
| gtcgagtcga tgaaagccgc atgtccggaa ttaggtggta | 1000 |

| | |
|---|---|
| <210> | 4 |
| <211> | 1000 |
| <212> | DNA |
| <213> | Bacteriophage KCCM 10977P |
| <400> | 4 |

| | |
|---|---|
| gtgatgaacc acaattggtt agaagacagg aacccctgtt taccgccttt gatgttcggc | 60 |
| tcggcgtatt ggttcccgat ttcatcatag tacgagttga tccataccaa aacgaatttc | 120 |
| ttttcagtga ccaacggggt gataacacgc caaaaactat tgagagcgcg agcgcgggtc | 180 |
| atatcttgtg tgtctttgcc cgcgatggca tcatcaactt ctttggtaga cggcaactgg | 240 |
| ctgattgagt caatgaatac gatgatcttg tcacctttct gagcatcatt cagaagctgt | 300 |
| gtcagcttga tcctcgtctc ttcaacgttt tcaatcggca gatacaagac acggtccatg | 360 |
| tcaataccca tagatgtcca atagttttca ttcgcaccgc cttcggaatc cgcgaagata | 420 |
| caaattgcat caggaaactt atccatgtaa gccttaacat ccaccagccc aaacatggtt | 480 |
| ttgaatgtac gagaatcccc caccaactgt ttgatgcctg atatcagacc accatcaata | 540 |
| cgaccggacc aggccaaatt cagaatagga atacccgtac tgcaaataat gtcaggcttc | 600 |
| agcgcatcgg tctttgacag cacttcggca ttcgggtcca gtttctttgc tgtcttgagc | 660 |
| atgcgagcca tcaatgaatc ggccatttcg tttcctcttg cttgttgatc gtaattaata | 720 |
| aatcggtgcc caagactttc ttggaaaata tattgattgc ttcgtgaatc gccattattg | 780 |
| acgggagttt ttcatcgtca atttcggaac ccccgcgttc tgttaaatac atattacgca | 840 |
| gacgattgtg ctgtgcccta ttgacacaag aaacattcaa tgcgatattc aggatataca | 900 |
| tcatttgttc agttgtaaca tcctttggaa tagcatgaac ataatatatc gcatcttcaa | 960 |
| aatagatatg ctgcaatgac tctggaattt cttccccgcc | 1000 |

| | |
|---|---|
| <210> | 5 |
| <211> | 21 |
| <212> | DNA |
| <213> | Artificial Sequence |
| <220> | |
| <223> | primer |
| <400> | 5 |

| | |
|---|---|
| ccctgctggc ttataagcaa c | 21 |

| | |
|---|---|
| <210> | 6 |
| <211> | 21 |
| <212> | DNA |
| <213> | Artificial Sequence |
| <220> | |
| <223> | primer |
| <400> | 6 |

| | |
|---|---|
| cgtgtgccat gatcatgaaa c | 21 |

| | |
|---|---|
| <210> | 7 |
| <211> | 21 |
| <212> | DNA |
| <213> | Artificial Sequence |
| <220> | |
| <223> | primer |
| <400> | 7 |

| | |
|---|---|
| aatgtcggca atagcgataa c | 21 |

| | |
|---|---|
| <210> | 8 |
| <211> | 21 |
| <212> | DNA |
| <213> | Artificial Sequence |
| <220> | |
| <223> | primer |
| <400> | 8 |

| | |
|---|---|
| cctctaagga cgttgcgcga a | 21 |

| | |
|---|---|
| <210> | 9 |
| <211> | 21 |
| <212> | DNA |
| <213> | Artificial Sequence |
| <220> | |
| <223> | primer |
| <400> | 9 |

| | |
|---|---|
| aattatgcaa atggccagca g | 21 |

| | |
|---|---|
| <210> | 10 |
| <211> | 21 |
| <212> | DNA |
| <213> | Artificial Sequence |
| <220> | |
| <223> | primer |
| <400> | 10 |

| | |
|---|---|
| taccacctaa ttccggacat g | 21 |

| | |
|---|---|
| <210> | 11 |
| <211> | 21 |
| <212> | DNA |
| <213> | Artificial Sequence |
| <220> | |
| <223> | primer |
| <400> | 11 |

| | |
|---|---|
| gtgatgaacc acaatcggtt a | 21 |

| | |
|---|---|
| <210> | 12 |
| <211> | 21 |
| <212> | DNA |
| <213> | Artificial Sequence |
| <220> | |
| <223> | primer |
| <400> | 12 |

| | |
|---|---|
| ggcggggaag aaattccaga g | 21 |

What is claimed is:

1. A novel bacteriophage that has a specific bactericidal activity against one or more Salmonella bacteria selected from the group consisting of Salmonella Enteritidis, Salmonella Typhimurium, Salmonella Gallinarum, and Salmonella Pullorum, that belong to the morphotype group of the family Myoviridae, and that has a total genome size of 157-159 kbp and major structural proteins with sizes of 44-46 kDa, 61-63 kDa and 79-81 kDa.

2. The bacteriophage according to claim 1, wherein the bacteriophage is identified by accession number KCCM10977P.

3. The bacteriophage according to claim 1, wherein the bacteriophage has the morphology depicted in FIG. 1.

4. The bacteriophage according to claim 1, wherein the bacteriophage includes one or more nucleic acid molecules selected from the group consisting of SEQ ID NO. 1, 2, 3, and 4 within the entire genome.

5. The bacteriophage according to claim 1, wherein each PCR product has a size of 1 kbp, upon performing PCR using one or more primer sets selected from the group consisting of SEQ ID NOs. 5 and 6, SEQ ID NOs. 7 and 8, SEQ ID NOs. 9 and 10, and SEQ ID NOs. 11 and 12.

6. The bacteriophage according to claim 1, wherein the bacteriophage has one or more properties of the following 1)-3):
1) acid-resistance in a pH range from pH 3.5 to pH 9.0;
2) heat-resistance in a temperature range from 37° C. to 60° C.; and
3) dry-resistance at 37-60° C. for 0-120 minutes.

7. A composition for the prevention or treatment of infectious diseases caused by one or more Salmonella bacteria selected from the group consisting of Salmonella Gallinarum, Salmonella Pullorum, Salmonella Typhimurium, and Salmonella Enteritidis, comprising the bacteriophage of any one of claims 1 to 6 as an active ingredient.

8. The composition according to claim 7, wherein the infectious disease caused by Salmonella Enteritidis or Salmonella Typhimurium is salmonellosis or Salmonella food poisoning, the infectious disease caused by Salmonella Gallinarum is Fowl Typhoid, and the infectious disease caused by Salmonella Pullorum is Pullorum disease.

9. The composition according to claim 7, wherein the composition is used as an antibiotic.

10. An animal feed or drinking water, comprising the bacteriophage of any one of claims 1 to 6 as an active ingredient.

11. A sanitizer or cleaner, comprising the bacteriophage of any one of claims 1 to 6 as an active ingredient.

12. A method for preventing or treating infectious diseases caused by one or more Salmonella bacteria selected from the group consisting of Salmonella Enteritidis, Salmonella Typhimurium, Salmonella Gallinarum, and Salmonella Pullorum, using the bacteriophage of claims 1 to 6.

13. A method for preventing or treating infectious diseases caused by one or more Salmonella bacteria selected from the group consisting of Salmonella Enteritidis, Salmonella Typhimurium, Salmonella Gallinarum, and Salmonella Pullorum, using the composition of claim 7.


SEQUENCE LISTING (12 sequences)

SEQ ID NO: 1 - DNA, 1000 bp, Bacteriophage KCCM 10977P
ccctgctggcttataagcaactcgatcagcttgtaaacaatctggctggtatcagcttggctgatttcgaaaggatctctatcggcatccagagggatatattaggcaatgataacctgacggcgtctgagaagagtagtctgttgggattgttacaggatgttgtcaaaaactaaaaagcccccgaaggggctttagtgaaattagtcttgcttcaggaactgctcgaactcatcaatggaagccgtctgtttcgcatcggcaccaccattattggctggaacagattgctgtgcattagaaggctgagattgttgttggtttagactttcctgcgctgttgggcgctggggttcctgagactgggtaggcgcatgtgccatagtagaagcaccaccttcaaccagaggctgattatcagggatggccagaactttgcgcaaacgtttttccagatcttcgtacgatttgaagttggccggattaaagaactcaaacagactgtgttctttttcccagatctcttcaatgtattcgtctgtccccaaaggtgccggagtatcccacttcacattggtgaagttggccaccagacctttccagttgccgaactctttctcttcgccaaagaggttcagaatcaggttcgcgccttcccacatatcgaacgggtcgaatttagggtcagttgagaacttaggattctgagccgaatccaggattttcttgacggcattaccgaactcaagcaagaagaccttgccgttgttttccggattgttgccatctttgatcaccaggatgttggcgtagtatttggtgtccggcagacgttttttgagaactgttttcagcttttcatcattcgtttctttctgttgtgcccacagaggacggtcatggtcacgaacaggatcatcgttaccgaaagtctgaggagagttttcgatataccaaccaccagcaccctggaatgcgtgtttcatgatcatggcacacg

SEQ ID NO: 2 - DNA, 1000 bp, Bacteriophage KCCM 10977P
aatgtcggcaatagcgataactgtactggaatcgttaacagtgcgcaactttttaccaggtgccagaacgatagaggggcagatggtttcaaagttagccagcagttgtaaagtgcgttcggagagagtgatctcttgcattagttgtatcctcaaaatatagtggggttcaagtcatatttgacgcaaattagtatcgcgtgtttgtagttatagaacaagtgataaattgccctacgcgcgataaataaatgcctgacggcatttataatattctgttttaataaaacctttctttatcagtctactcgcttcgctcgtgataatactcgttgctcgcaaagctcacaactcgtatattacgcacggattgttcaacaagaaagcgatttttattcaacaagtaaaatattttatttggtctaaacagagcatgacattattatgtagccaagtttgctaacacgtgagaaataatatatgaagcaatttgttggtttatacgcagtaggggaagaccaagaagcaattctttccatagcagaacaacgttcgtcattaaaaggcgtttatttacaaagccttttccgtacatcggggtttattgtgtcaccgatgttggtgataccattactcccaaataacaaaggtctgtatgttggcattattcaacaaggccaggcgcgggaagtgaaagttgttccactgctggcatctaatgaagaattgttttctcagattcttgagccgaaagtactacaacaatgtattggcacgatcgactgtttatttggttccaacaaagaaggcgaggcaacccccgcctatgtgaatcaagatatttgaaatggttagagcgccacttttttcattttaacagggtggcgctccataagataaaatttatatctctcatgagaatgcctgagagcgtggttgtaggaaccgttgtagcgcaggttgtctaccaggtcccagattcgcgcaacatccttagagg

SEQ ID NO: 3 - DNA, 1000 bp, Bacteriophage KCCM 10977P
aattatgcaaatggccagcagggcatcaatggcagcaaactccgtcaggccatctggctgatggttgagcacctcaaggccggaggctcccctgacatcatccatggcactgtcgttggtagtccgcagtcccctatggcgacagcagtctctcggcacttcggtggccacacaaccactgtactcggcgcgaccaaacccacgacatgtatgaaccacgacatggtggcaatgtcggcctggtttggtagtatattcaacttcgtcggctcgggatacaacagcaccatccagccgcggtgtaagaagttgatcgaacaacagaatccaaaggcgtattatctggagtatggtatcaccctcgaccatacggcccactcccctgagcgcattgctgggttccatatgctgggcggggagcaggttgccaacatccctgaccatattaccgatctaattatccctgctggctcctgcaactcatgtacaagcatcctgaccggactggcaatgcatccgaaaccaaatctgaagaatgtctatctgatcgggattggaccaaaccgattagatttcattgaaagtcgtttgcgcattatcggtaagcaagcaaacctccctcacataaccgatttcactcgtcgctatcacgacaacccagattatgtgtatggtaagaaggatcttcagcatgcctctaagagcgtttcgctggctggcctcctaagtggtatcaggcagaaggacgagccagaggtaacgcttcctcgctttgaggtacaccattgggatcttcataccactaattgggttcgttacaacgacctgatggactaccagtggggtgatatcgaactgcacccgcgctatgaaggcaaggtcatgacctggatccagcagcacaagccagagatgctgaacgagaacactctgttctggatcgttggtagtaagccatatgtcgagtcgatgaaagccgcatgtccggaattaggtggta

SEQ ID NO: 4 - DNA, 1000 bp, Bacteriophage KCCM 10977P
gtgatgaaccacaattggttagaagacaggaacccctgtttaccgcctttgatgttcggctcggcgtattggttcccgatttcatcatagtacgagttgatccataccaaaacgaatttcttttcagtgaccaacggggtgataacacgccaaaaactattgagagcgcgagcgcgggtcatatcttgtgtgtctttgcccgcgatggcatcatcaacttctttggtagacggcaactggctgattgagtcaatgaatacgatgatcttgtcacctttctgagcatcattcagaagctgtgtcagcttgatcttcgtcttttcaacgttttcaatcggcagatacaagacacggtccatgtcaatacccatagatgtccaatagttttcattcgcaccgccttcggaatccgcgaagatacaaattgcatcaggaaacttatccatgtaagccttaacatccaccagcccaaacatggttttgaatgtacgagaatcccccaccaactgtttgatgcctgatatcagaccaccatcaatacgaccggaccaggccaaattcagaataggaatacccgtactgcaaataatgtcaggcttcagcgcatcggtctttgacagcacttcggcattcgggtccagtttctttgctgtcttgagcatgcgagccatcaatgaatcggccatttcgtttcctcttgcttgttgatcgtaattaataaatcggtgcccaagactttcttggaaaatatattgattgcttcgtgaatcgccattattgacgggagtttttcatcgtcaatttcggaacccccgcgttctgttaaatacatattacgcagacgattgtgctgtgccctattgacacaagaaacattcaatgcgatattcaggatatacatcatttgttcagttgtaacatcctttggaatagcatgaacataatatatcgcatcttcaaaatagatatgctgcaatgactctggaatttcttccccgcc

SEQ ID NO: 5 - DNA, 21 bp, Artificial Sequence
  primer
ccctgctggcttataagcaac

SEQ ID NO: 6 - DNA, 21 bp, Artificial Sequence
  primer
cgtgtgccatgatcatgaaac

SEQ ID NO: 7 - DNA, 21 bp, Artificial Sequence
  primer
aatgtcggcaatagcgataac

SEQ ID NO: 8 - DNA, 21 bp, Artificial Sequence
  primer
cctctaaggatgttgcgcgaa

SEQ ID NO: 9 - DNA, 21 bp, Artificial Sequence
  primer
aattatgcaaatggccagcag

SEQ ID NO: 10 - DNA, 21 bp, Artificial Sequence
  primer
taccacctaattccggacatg

SEQ ID NO: 11 - DNA, 21 bp, Artificial Sequence
  primer
gtgatgaaccacaattggtta

SEQ ID NO: 12 - DNA, 21 bp, Artificial Sequence
  primer
ggcggggaagaaattccagag